(19) United States
(12) Patent Application Publication
(10) Pub. No.: US 2024/0116998 A1
(43) Pub. Date: Apr. 11, 2024

(54) GLUCAGON-LIKE PEPTIDE-1 RECEPTOR ANTAGONISTS

(71) Applicant: INDIANA UNIVERSITY RESEARCH AND TECHNOLOGY CORPORATION, Bloomington, IN (US)

(72) Inventors: Richard D. DIMARCHI, Carmel, IN (US); Piotr Andrzej MROZ, Bloomington, IN (US); Kishore THALLURI, Bloomington, IN (US)

(21) Appl. No.: 18/264,157

(22) PCT Filed: Feb. 15, 2022
(86) PCT No.: PCT/US2022/016406

§ 371 Date: Aug. 3, 2023

Related U.S. Application Data
(60) Provisional application No. 63/149,852, filed on Feb. 16, 2021.

(51) Int. Cl. C07K 14/605; A61P 3/08

(52) U.S. Cl. CPC C07K 14/605; A61P 3/08; A61K 38/00

(57) ABSTRACT

Provided herein are GLP-1 receptor antagonists peptides and pharmaceutical compositions for the treatment of hypoglycemia. Further provided herein are methods of treating atypical hypoglycemia in patients that have become hyperinsulinemic, including those who become hyperinsulinemic after bariatric surgery.

### CROSS REFERENCE TO RELATED APPLICATIONS

This application claims priority to U.S. Provisional Patent Application No. 63/149,852 filed on Feb. 16, 2021, the disclosure of which is expressly incorporated herein.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 59 kilobytes acii (text) file named “333142seqlist_ST25.txt,” created on Feb. 14, 2022.

### BACKGROUND

Glucagon-like peptide-1 (GLP-1) plays an important role in regulating blood glucose levels in humans. Its actions include stimulation of insulin synthesis and secretion, inhibition of glucagon secretion, and inhibition of food intake. Normally, the body maintains the concentration of glucose in the blood within a range of about 70 to 110 milligrams per deciliter (mg/dL), or 3.9 to 6 1 millimoles per liter (mmol/L). However, conditions can arise where glucose level becomes too low, leading to hypoglycemia. Hypoglycemia is most often caused by drugs taken to control diabetes. Much fewer common causes of hypoglycemia are referred to herein as “atypical hypoglycemia”, and can occur independent of exogenous insulin administration.

Atypical hypoglycemia can occur in people who drink heavily without eating, as alcohol can block the formation of glucose in the liver. In addition, people with advanced liver disease, such as viral hepatitis, cirrhosis, or cancer of the liver may not be able to store and produce sufficient glucose. Atypical hypoglycemia can also result in infants and children that have a congenital mutation that render them hyperinsulinemic.

More recently, over the course of the last decade there has been an increased awareness that atypical hypoglycemia is a complication that can arise after surgical procedures performed for the purpose of reversing extreme forms of obesity. Some patients (less than 10%) receiving Roux-en-Y gastric bypass (RYGB) surgeries subsequently develop hyperinsulinemic hypoglycemia, wherein blood glucose concentrations can become low enough (20-40 mg/dL) to cause seizures, altered mental status, loss of consciousness, cognitive dysfunction, disability, and death.

One approach to treating hyperinsulinemic-induced hypoglycemia is to administer a GLP-1 receptor antagonist. Such peptide antagonists block the ability of inappropriately elevated concentrations of plasma GLP-1 to stimulate insulin secretion, and function to normalize plasma glucose and reduce risk of cognitive impairment, vascular disease and potentially sudden death in patients experiencing atypical hypoglycemia.

Exendin-4 (SEQ ID NO: 1) is a 39 amino acid agonist of the glucagon-like peptide 1 (GLP-1) receptor. Exendin-4 is present in the saliva of the Gila monster, Heloderma suspectum. Ex-4 (9-39)a (SEQ ID NO: 2) is an N-terminal truncated derivative of Exendin-4 that is known to function as a GLP-1 receptor antagonist. However, Ex-4 (9-39)a suffers from two notable limitations regarding its potential use to treat chronic atypical hypoglycemia: its nonhuman amino acid sequence, and its relatively short in vivo duration of action.

In accordance with one embodiment of the present disclosure a set of novel optimized GLP-1 antagonists are provided for use as drug candidates in treatment of atypical hypoglycemia, including hyperinsulinemic induced hypoglycemia resulting after post-bariatric surgical procedures or resulting from congenital mutations.

### SUMMARY

As disclosed herein compositions and methods are provided for treating patients experiencing atypical hypoglycemia, and more particularly in one embodiment, treating patients who experience hyperinsulinemia induced hypoglycemia. In accordance with one embodiment compositions and methods are provided for treating hyperinsulinemia induced hypoglycemia resulting after post-bariatric surgery. In one embodiment the method comprises the administration of a glucagon-like peptide-1 receptor antagonist (GLP1RA) in an amount effective to elevate blood glucose levels and alleviate associated acute symptoms and chronic outcomes associated with hypoglycemia.

In accordance with one embodiment a GLP-1 receptor antagonist peptide is provided comprising the amino acid sequence
DX₁₀X₁₁RYLX₁₅X₁₆QAVREFX₂₃EWLVRGGPSSGAPPPSX₄₀R₂₀ (SEQ ID NO: 5), wherein
X₁₀ is Trp, dTrp or Val;
X₁₁ is Trp, dTrp or Ser;
X₁₅ is Glu or dGlu
X₁₆ is Trp, dTrp, dGlu or Glu;
X₂₃ is Ile or dIle;
X₄₀ is an acylated amino acid, and
R₂₀ is COOH or CONH₂, optionally wherein the peptide comprises one or more substitutions of Aib at any of positions 16, 18, 19, 24, 26 or 28, relative to the native Exendin4 sequence (SEQ ID NO: 1), or optionally a substitution of Lys at position 12, relative to the native Exendin4 sequence (SEQ ID NO: 1), optionally with the proviso that when one of X₁₀ or X₁₁ is Trp or dTrp, the other is not Trp or dTrp.

In accordance with one embodiment a GLP-1 receptor antagonist peptide is provided having the amino acid sequence of

(SEQ ID NO: 96)
DVWRYLX₁₅EQAVREFIEWLVRGGPSSGAPPPSX₄₀ R₂₀,

wherein
X₁₅ is dGlu;
X₄₀ is an acylated Lys and R₂₀ is COOH or CONH₂, wherein the acyl group of the acylated Lys is a C16-C18 acid or diacid, optionally linked via a spacer to the Lys side chain. In one embodiment the spacer comprises a minipeg or a gamma glutamic acid subunit or any multiple or combination of such minipeg or gamma Glu molecules. In one embodiment the peptide of SEQ ID NO: 96 is modified with 1, 2 or 3 amino acid substitutions, including for example, substitution with amino isobutyric acid (Aib) at one or more of positions 16, 18, 19, 24, 26 or 28, relative to the native Exendin4 sequence of SEQ ID NO: 1; or substitution of Lys at position 12.

In accordance with one embodiment a GLP-1 receptor antagonist peptide is provided comprising the amino acid sequence
DX₁₀X₁₁RYLX₁₅X₁₆QAVREFX₂₃EWLVRGGPSSGAPPPSX₄₀R₂₀ (SEQ ID NO: 5), wherein
X₁₀ is Trp, dTrp or Val;
X₁₁ is Trp, dTrp or Ser;
X₁₅ is Glu or dGlu
X₁₆ is Trp, dTrp, dGlu or Glu;
X₂₃ is Ile or dIle;
X₄₀ is an acylated amino acid, and R₂₀ is COOH or CONH₂-, optionally wherein the peptide comprises one or more substitutions of Aib at any of positions 16, 18, 19, 24, 26 or 28, relative to the native Exendin4 sequence (SEQ ID NO: 1), or optionally an N-terminal extension of the peptide of SEQ ID NO: 5 by X₇X₈-, wherein X₇ is an acylated amino acid (e.g., Lys) and X₈ is Gly or C₁-C₃ N-alkyl Gly, with position numbering relative to the native Exendin4 sequence (SEQ ID NO: 1), optionally with the proviso that when one of X₁₀ or X₁₁ is Trp or dTrp, the other is not Trp or dTrp.

In accordance with one embodiment a GLP-1 receptor antagonist peptide is provided having the amino acid sequence of

(SEQ ID NO: 96)
DVWRYLX₁₅EQAVREFIEWLVRGGPSSGAPPPSX₄₀ R₂₀,

wherein
X₁₅ is dGlu;
X₄₀ is an acylated Lys and R₂₀ is COOH or CONH₂-, wherein the acyl group of the acylated Lys is a C16-C18 acid or diacid, optionally linked via a spacer to the Lys side chain. In one embodiment the spacer comprises a minipeg or a gamma glutamic acid subunit or any multiple or combination of such minipeg or gamma Glu molecules. In one embodiment the peptide of SEQ ID NO: 96 is modified with 1, 2 or 3 amino acid substitutions, including for example, substitution with amino isobutyric acid (Aib) at one or more of positions 16, 18, 19, 24, 26 or 28, relative to the native Exendin4 sequence of SEQ ID NO: 1; or by the addition of a dipeptide X₇X₈ to the N-terminus of SEQ ID NO: 96, wherein X₇ is an acylated amino acid (e.g., Lys) and X₈ is Gly or C₁-C₃ N-alkyl Gly, with position numbering relative to the native Exendin4 sequence (SEQ ID NO: 1).

In one embodiment 1 to 3 amino acids are added to the N-terminus of the peptide of SEQ ID NO: 5 or an analog thereof. In one embodiment one of the amino acids comprising the N-terminal extension is an acylated amino acid. In one embodiment the N-terminal extension is a dipeptide, X₇X₈-, wherein X₇ is an acylated lysine, optionally wherein the lysine is in the D-conformation and X₈ is any amino acid. In one embodiment the N-terminal extension is a self-cleaving dipeptide linked to the N-terminal alpha amine of a 9-29 exendin4 analog (e.g., a peptide of SEQ ID NO: 5) to form a prodrug of any of the GLP-1 antagonist of the present disclose. One advantage of using a prodrug derivative of a GLP-1 antagonist is that such an approach extends peptide's biological half-life based on a strategy of inhibiting recognition of the prodrug by the GLP-1 receptor. In one embodiment the prodrug derivative comprises a self-cleaving dipeptide (A-B) covalently linked to the GLP-1 antagonist wherein the dipeptide is cleaved under physiological conditions and in the absence of enzymatic activity to restore full activity to the GLP-1 antagonist. In one embodiment the GLP-1 antagonist is modified by the covalent linkage of one or more dipeptides (A-B) to an amine of GLP-1 antagonist, wherein A is an amino acid or a hydroxy acid and B is an N-alkylated amino acid linked to the GLP-1 antagonist through an amide bond between a carboxyl moiety of B and an amine of the GLP-1 antagonist. In one embodiment, A-B comprises the structure:

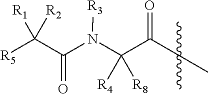

linked to the GLP-1 antagonist through an amide bond between the carboxyl of A-B and an amine of the GLP-1 antagonist wherein
R₁-, R₂-, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃-, (C₁-C₄ alkyl)CONH₂-, (C₁-C₄ alkyl)COOH, (C₁-C₄ alkyl)NH_{2,} (C₁-C₄ alkyl)NHC(NH₂⁺)NH₂-, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇-, (C₁-C₄ alkyl)(C₃-C₉ heteroaryl), and C₁-C₁₂ alkyl(W1)C₁-C₁₂ alkyl, wherein W1 is a heteroatom selected from the group consisting of N, S and O;
R₃ is selected from the group consisting of C₁-C₁₈ alkyl, (C₁-C₁₈ alkyl)OH, (C₁-C₁₈ alkyl)NH₂-, (C₁-C₁₈ alkyl)SH, (C-C₄ alkyl)(C₃-C₆)cycloalkyl, (C₀-C₄ alkyl)(C₂-C₅ heterocyclic), (C₀-C₄ alkyl)(C₆-C₁₀ aryl)R₇-, and (C₁-C₄ alkyl)(C₃-C₉ heteroaryl) or R₄ and R₃ together with the atoms to which they are attached form a pyrrolidine ring;
R₅ is NHR₆ or OH;
R₆ is H, C₁-C₈ alkyl; and
R₇ is selected from the group consisting of H and OH, wherein the chemical cleavage half-life (t_{1/2}) of A-B from said GLP-1 antagonist is at least about 1 hour to about 1 week in PBS under physiological conditions. In one embodiment the dipeptide A-B is covalently linked to the N-terminal alpha amine of the GLP-1 antagonist amino acid sequence. In one embodiment wherein R₁ is H, C₁-C₄ alkyl, (C₁-C₄ alkyl)OH or (C₁-C₄ alkyl)NH₂-; R₂ is H, R₃ is C₁-C₆ alkyl; R₄ is H, C₁-C₄ alkyl, or (CH₂)(C₆ aryl)R₇-; R₈ is NH₂-; R₂ is H or OH and R₈ is hydrogen.

The present disclosure moreover provides pharmaceutical compositions comprising any of the GLP-1 antagonist peptides and variant peptides described herein and a pharmaceutically acceptable carrier, diluent, or excipient. In one embodiment a method of treating a patient suffering from atypical hypoglycemia is provided wherein the method comprises the step of administering to a patient in need thereof a pharmaceutical composition comprising a GLP-1 antagonist peptide or variant peptides described herein in an amount effective to elevate blood glucose levels.

In accordance with one embodiment a method of treating atypical hypoglycemia is provided wherein the method comprises the steps of administering a GLP-1 receptor antagonist peptide having the amino acid sequence of
DVX₁₁RYLX₁₅X₁₆QAVREFX₂₃EWLVRGGPSSGAPPPSK (SEQ ID NO: 11), wherein X₁₁ is Trp, or dTrp; X₁₅ is Glu, or dGlu; X₁₆ is Glu, or dGlu; and X₂₃ is Ile, or dIle; in an amount therapeutically effective for increasing blood glucose levels. In one embodiment the GLP-1 receptor antagonist is acylated with a fatty acid or diacid group of sufficient size to bind serum albumin with high affinity.

In one embodiment a GLP-1 receptor antagonist is provided, wherein the antagonist comprises the amino acid sequence of

(SEQ ID NO: 98)
DX₁₀X₁₁X₁₂YLX₁₅X₁₆QAVREFX₂₃X₂₄WLVRGGPSSGAPPPS;

wherein
X₁₀ is Trp, dTrp or Val;
X₁₁ is Trp, dTrp or Ser;
X₁₂ is Arg, Lys or Ser;
X₁₅ is Glu or dGlu;
X₁₆ is Trp, dTrp, dGlu or Glu;
X₂₃ is Ile or dIle;
X₂₄ is Ala or Glu; and
said GLP-1 receptor antagonist is acylated with a fatty acid or diacid group of sufficient size to bind serum albumin with high affinity, optionally wherein an amino acid of the GLP-1 receptor antagonist is acylated with a C16-C18 fatty acid or C16-C18 fatty diacid, optionally with the proviso that only one of X₁₀-, X₁₁ or X₁₆ is Trp or dTrp.

In one embodiment a GLP-1 receptor antagonist is provided wherin the antagonist comprises the amino acid sequence of

(SEQ ID NO: 98)
DX₁₀X₁₁X₁₂YLX₁₅X₁₆QAVREFX₂₃X₂₄WLVRGGPSSGAPPPS;

wherein
X₁₀ is Trp, dTrp or Val;
X₁₁ is Trp, dTrp or Ser;
X₁₂ is Arg, Lys or Ser;
X₁₅ is Glu or dGlu;
X₁₆ is Trp, dTrp, dGlu or Glu;
X₂₃ is Ile or dIle;
X₂₄ is Ala or Glu, further wherein the GLP-1 receptor antagonist is acylated with a fatty acid or diacid group of sufficient size to bind serum albumin with high affinity, optionally wherein an amino acid of the GLP-1 receptor antagonist is acylated with a C16-C18 fatty acid or C16-C18 fatty diacid. In a further embodiment a peptide of SEQ ID NO: 98 is provided wherein X₁₀ is Val, X₁₁ is Trp, dTrp or Ser, X₁₂ is Arg, X₁₅ is Glu or dGlu, X₁₆ is dGlu or Glu, X₂₃ is Ile or dIle, and X₂₄ is Ala or Glu, optionally wherein X₁₀ is Val, X₁₁ is Ser, X₁₂ is Arg, X₁₅ is Glu or dGlu, X₁₆ is dGlu or Glu, X₂₃ is Ile, and X₂₄ is Ala, optionally wherein X₁₀ is Val, X₁₁ is Ser, X₁₂ is Arg, X₁₅ is Glu, X₁₆ is Glu, X₂₃ is Ile, and X₂₄ is Ala.

In one embodiment the GLP-1 receptor antagonists of the present disclosure are acylated with a fatty acid or diacid group of sufficient size to bind serum albumin with high affinity, optionally wherein the acylated amino acid is the C-terminal amino acid, and optionally further modified by the linkage of a self-cleaving dipeptide via an amide bond, wherein an amino acid of the dipeptide is optionally acylated with a fatty-acyl group of sufficient size to bind serum albumin with high affinity. In one embodiment an acylated amino acid is added to the C-terminus of SEQ ID NO: 11 at position 40 and optionally the added acylated amino acid is Lys acylated with a C14-C20 fatty acid or fatty diacid, optionally linked to the amino acid side chain via any of the spacers disclosed herein.

In one embodiment the pharmaceutical compositions for treating atypical hypoglycemia comprise any of the GLP-1 receptor antagonist disclosed herein in combination with any existing therapeutics useful for treating hypoglycemia. For example, the pharmaceutical composition may include a GLP-1 receptor antagonist of the present invention and one or more of the following: glucose supplements (e.g., dextrose); glucose-elevating agents such as glucagon and glucagon analogs and inhibitors of insulin secretion (e.g., diazoxide, octreotide).

### BRIEF DESCRIPTION OF THE DRAWINGS

FIG. 1 is a graph presenting the dose dependent change in blood glucose levels over time after subcutaneous administration of a vehicle control, Ex-4 (a GLP-1 agonist having an amino acid sequence of SEQ ID NO: 1), or a GLP-1 antagonist peptide (DVSRYLEEQAVREFIEWLVRGGPSSGAPPPSK₄₀[mPEG-γE-C16]acid; SEQ ID NO: 16 to mice followed by intraperitoneal administration of glucose (1.5 g glucose per kg body weight), wherein the glucose is administered subcutaneously 4 hr after the GLP-1 antagonist.

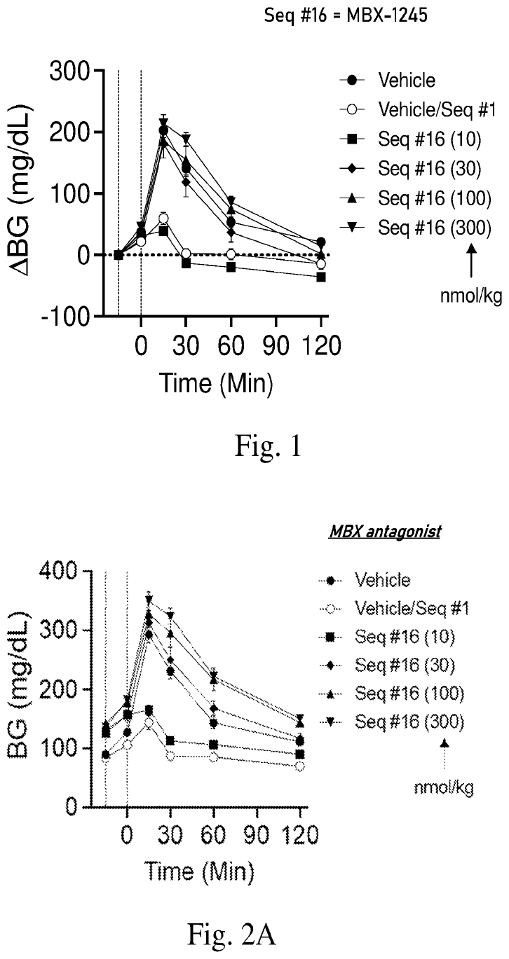

FIGS. 2A & 2B present data from a glucose tolerance test wherein mice are subcutaneously administered the GLP-1 antagonist followed four hours later by intraperitoneal administration of glucose (1.5 g glucose per kg body weight). The results demonstrate that the GLP-1 antagonist of SEQ ID NO: 16 (FIG. 2A) is a full antagonist and has a much higher potency than the GLP-1 antagonist Ex-9-40 (DVSKQMEEEAVRLFIEWLKNGGPSSGAPPPS; SEQ ID NO: 2); FIG. 2B).

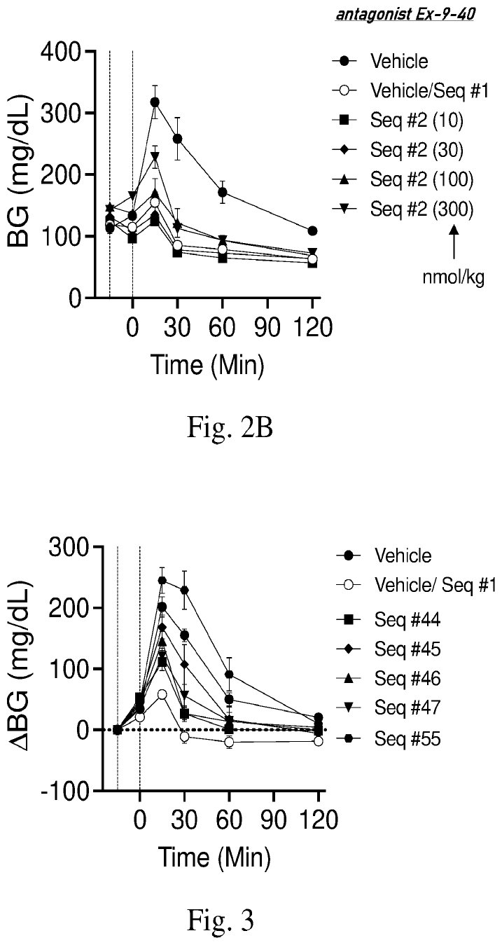

FIG. 3 presents data from a glucose tolerance test investigating the effect of Aib substitutions on the efficacy of the GLP-1 antagonists. Mice are subcutaneously administered the GLP-1 antagonist followed four hours later by intraperitoneal administration of glucose (1.5 g glucose per kg body weight). FIG. 3 demonstrates the activity of a GLP-1 antagonist peptide analog of 9-40Jant4-K₄₀ (DVSSYLEEQAVREFIAWLVKGGPSSGAPPPSK; SEQ ID NO: 3) that comprises the sequence of(SEQ ID NO: 47), and derivatives thereof comprising a series of Aib substitutions s SEQ ID NO: 43 (Aib at postion 20), SEQ ID NO: 44 (Aib at postion 18), and, SEQ ID NO: 45 (Aib at postion 11), SEQ ID NO: 46 (Aib at postion 10).

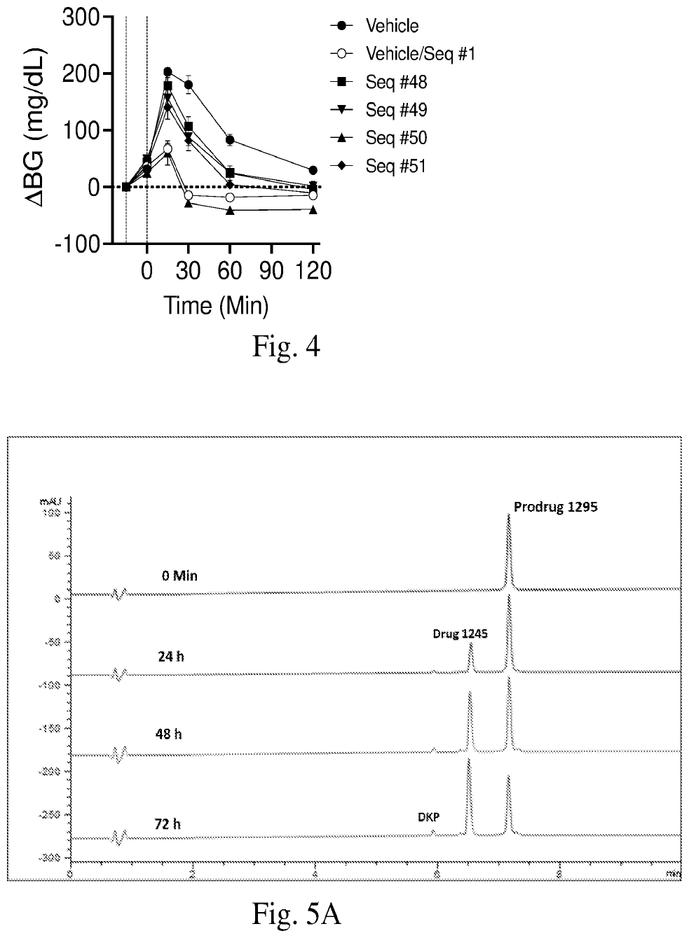

FIG. 4 presents data from a glucose tolerance test investigating the effect of D-amino acid substitutions on the efficacy of the GLP-1 antagonists. Mice are subcutaneously administered the GLP-1 antagonist followed four hours later by intraperitoneal administration of glucose (1.5 g glucose per kg body weight). The GLP-1 antagonist activity of an (miniPEG)₂-γE-C16 acylated 9-40Jant4-K₄₀ (DVSSYLEEQAVREFIAWLVKGGPSSGAPPPSX₄₀ (SEQ ID NO: 12)) peptide SEQ ID NO: 48 wherein X₄₀ is Lys acylated with (miniPEG)₂-gamma Glu-C16 and has a C-terminal amide) relative to vehicle control and variants comprising a substitution wherein X₄₀ is Lys acylated with C16 and a C-terminal amide with the corresponding amino acid in the D-configuration as follows: SEQ ID NO: 49 (d-Glu¹⁵), SEQ ID NO: 50 (d-Val¹⁹), SEQ ID NO: 51 (d-Ile²³).

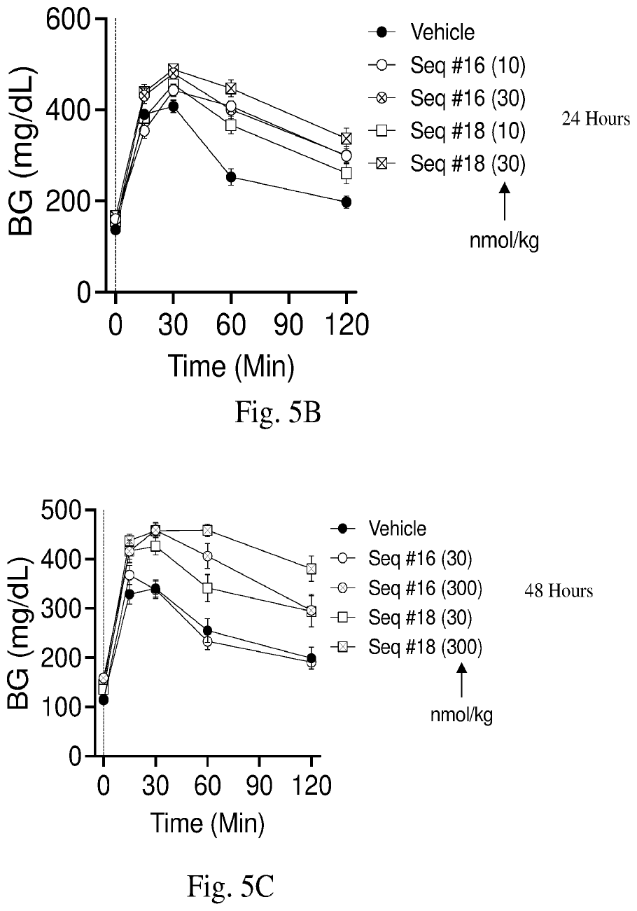

FIGS. 5A-5D provide data on prodrug derivatives of GLP-1 receptor antagonists of the present invention. FIG. 5A presents mass spectrophotometer data for the prodrug dK⁷ (mPeg-γE-diacid C18) N-Me-Gly⁸ peptide SEQ ID NO: 19 incubated in PBS at 37° C. over time. FIGS. 5B-5D are graphs presenting the GLP-1 antagonist activity of a dipeptide prodrug of DVSRYLEEQAVREFIEWLVRGGPSSGAPPPSX₄₀-; SEQ ID NO: 18, wherein the peptide has been modified with a covalently linked dK⁷ (mPeg-γE-diacid C18) N-iPr-Gly⁸ dipeptide, in a glucose tolerance test, wherein a glucose challenge is administered 24 h (FIG. 5B), 48 h (FIG. 5C) or 120 h (FIG. 5D) after administration of the antagonist.

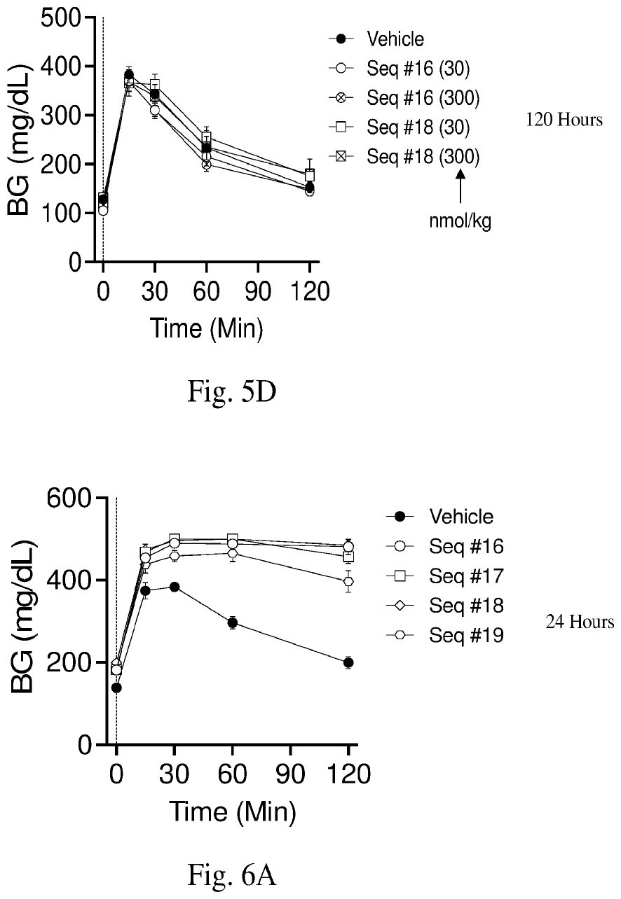

FIGS. 6A & 6B provide data on derivatives of GLP-1 receptor antagonists of the present invention comprising two acylated amino acids. FIGS. 6A and 6B are graphs presenting the results of a glucose tolerance test, wherein a glucose challenge is administered 24 h (FIG. 6A) or 48 h (FIG. 6B) after administration of the antagonist, wherein

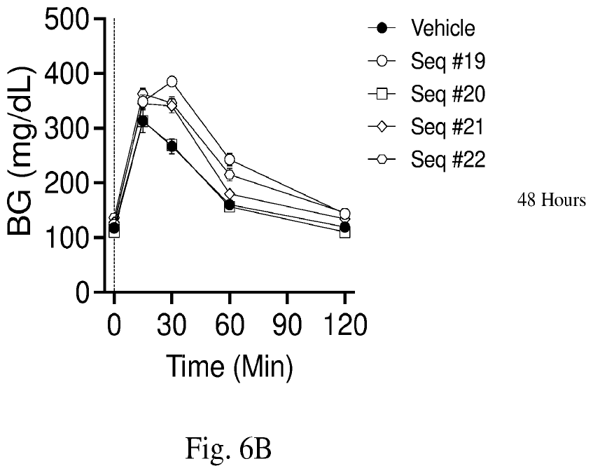

(SEQ ID NO: 16)
SEQ ID NO: 16 is R¹², E²⁴, R²⁸, K⁴⁰
[mPEG-γE-C16]-(Jant4, 9-40)acid;
(SEQ ID NO: 17)
SEQ ID NO: 17 is dK⁷(mPeg-γE(C18-diacid)G⁸,
R¹², E²⁴, R²⁸, K⁴⁰[mPEG-γE-C16]-
(Jant4, 9-40)acid;
(SEQ ID NO: 18)
SEQ ID NO: 18 dk⁷(mPeg-γE(C18-diacid)i-Pr,
G⁸, R¹², E²⁴, R²⁸, K⁴⁰[mPEG-γE-C16]-
(Jant4, 9-40)acid;
(SEQ ID NO: 19)
SEQ ID NO: 19 dK⁷(mPeg-γE(C18-diacid)N-Me,
G⁸, R¹², E²⁴, R²⁸ K⁴⁰[mPEG-γE-C16]-
(Jant4, 9-40)acid;
(SEQ ID NO: 20)
SEQ ID NO: 20 dk⁷(K(mPeg-γE-C18 diacid)₂
G⁸, R¹², E²⁴, R²⁸ K⁴⁰[mPEG-γE-C18 diacid]
(Jant4, 9-40)amide;
(SEQ ID NO: 21)
SEQ ID NO: 21 dK⁷(K(mPeg-γE-C18 diacid)₂
G⁸, R¹², E²⁴, R²⁸ K⁴⁰[mPEG-γE-C16]
(Jant4, 9-40)amide;
(SEQ ID NO: 22)
SEQ ID NO: 22 dK⁷(K(mPeg-γE-C18 diacid)₂
N-Me, G⁸, R¹², E²⁴, R²⁸) K⁴⁰[mPEG-γE-C16]
(Jant4, 9-40)amide.

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to designate any value or range of values to only this broader definition. Each value or range of values preceded by the term “about” is also intended to encompass the embodiment of the stated absolute value or range of values.

As used herein the term “amino acid” encompasses any molecule containing both amino and carboxyl functional groups, wherein the amino and carboxylate groups are attached to the same carbon (the alpha carbon). The alpha carbon optionally may have one or two further organic substituents. An amino acid can be designated by its three-letter code, one letter code, or in some cases by the name of its side chain. For example, a non-canonical amino acid comprising a cyclohexane group attached to the alpha carbon is termed “cyclohexane” or “cyclohexyl.” For the purposes of the present disclosure designation of an amino acid without specifying its stereochemistry is intended to encompass either the L or D form of the amino acid, or a racemic mixture. However, in the instance where an amino acid is designated by its three letter code (i.e., Lys), such a designation is intended to specify the native L form of the amino acid, whereas the D form will be specified by inclusion of a lower case d before the three letter code or single code (i.e., dLys or dK).

As used herein the term “hydroxyl acid” refers to amino acids that have been modified to replace the alpha carbon amino group with a hydroxyl group.

As used herein the term “non-coded amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

A “bioactive polypeptide” refers to polypeptides which can exert a biological effect in vitro and/or in vivo.

As used herein a general reference to a peptide is intended to encompass peptides that have modified amino and carboxy termini. For example, an amino acid sequence designating the standard amino acids is intended to encompass standard amino acids at the N- and C-terminus as well as a corresponding hydroxyl acid at the N-terminus and/or a corresponding C-terminal amino acid modified to comprise an amide group in place of the terminal carboxylic acid.

As used herein an “acylated” amino acid is an amino acid comprising an acyl group which is non-native to a naturally occurring amino acid, regardless by the means by which it is produced. Exemplary methods of producing acylated amino acids and acylated peptides are known in the art and include acylating an amino acid before inclusion in the peptide or peptide synthesis followed by chemical acylation of the peptide. In some embodiments, the acyl group causes the peptide to have one or more of (i) a prolonged half-life in circulation, (ii) a delayed onset of action, (iii) an extended duration of action, (iv) an improved resistance to proteases, and (v) increased potency at the GLP-1 receptor.

As used herein, an “alkylated” amino acid is an amino acid comprising an alkyl group which is non-native to a naturally occurring amino acid, regardless of the means by which it is produced. Exemplary methods of producing alkylated amino acids and alkylated peptides are known in the art and including alkylating an amino acid before inclusion in the peptide or peptide synthesis followed by chemical alkylation of the peptide. Without being held to any particular theory, it is believed that alkylation of peptides will achieve similar, if not the same, effects as acylation of the peptides, e.g., a prolonged half-life in circulation, a delayed onset of action, an extended duration of action, an improved resistance to proteases.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

As used herein, the term “hydrophilic moiety” refers to any compound that is readily water-soluble or readily absorbs water, and which are tolerated in vivo by mammalian species without toxic effects (i.e. are biocompatible). Examples of hydrophilic moieties include polyethylene glycol (PEG), polylactic acid, polyglycolic acid, a polylactic-polyglycolic acid copolymer, polyvinyl alcohol, polyvinylpyrrolidone, polymethoxazoline, polyethyloxazoline, polyhydroxyethyl methacrylate, polyhydroxypropyl methacrylamide, polymethacrylamide, polydimethylacrylamide, and derivatized celluloses such as hydroxymethylcellulose or hydroxyethylcellulose and co-polymers thereof, as well as natural polymers including, for example, albumin, heparin and dextran.

As used herein, the term “treating” includes alleviation of the symptoms associated with a specific disorder or condition and/or preventing or eliminating said symptoms. For example, as used herein the term “treating hypoglycemia” will refer in general to maintain or increase blood glucose levels to near normal levels.

As used herein an “effective” amount or a “therapeutically effective amount” of a GLP-1 receptor antagonist refers to a nontoxic but sufficient amount of a GLP-1 antagonist to provide the desired effect. For example, one desired effect would be the prevention or treatment of hypoglycemia. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as intranasal, inhalation, subcutaneous, intramuscular, intraspinal, or intravenous.

As used herein the term “derivative” is intended to encompass chemical modification to a compound (e.g., an amino acid), including chemical modification in vitro, e.g. by introducing a group in a side chain in one or more positions of a polypeptide, e.g. a nitro group in a tyrosine residue, or iodine in a tyrosine residue, or by conversion of a free carboxylic group to an ester group or to an amide group, or by converting an amino group to an amide by acylation, or by acylating a hydroxy group rendering an ester, or by alkylation of a primary amine rendering a secondary amine or linkage of a hydrophilic moiety to an amino acid side chain. Other derivatives are obtained by oxidation or reduction of the side-chains of the amino acid residues in the polypeptide.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein, the term “selectivity” of a molecule for a first receptor relative to a second receptor refers to the following ratio: EC₅₀ of the molecule at the second receptor divided by the EC₅₀ of the molecule at the first receptor. For example, a molecule that has an EC₅₀ of 1 nM at a first receptor and an EC₅₀ of 100 nM at a second receptor has 100-fold selectivity for the first receptor relative to the second receptor.

As used herein an amino acid “modification” refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, WI), ChemPep Inc. (Miami, FL), and Genzyme Pharmaceuticals (Cambridge, MA). Atypical amino acids may be purchased from commercial suppliers, synthesized de novo, or chemically modified or derivatized from naturally occurring amino acids.

As used herein an amino acid “substitution” refers to the replacement of one amino acid residue by a different amino acid residue.

As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln, cysteic acid and homocysteic acid;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine

Throughout the application, all references to a particular amino acid position by number (e.g., position 28) refer to the amino acid at that position in native Exendin4 (SEQ ID NO: 1) or the corresponding amino acid position in any analogs thereof. For example, a reference herein to “position 28” would mean the corresponding position 27 for an analog of Exendin4 in which the first amino acid of SEQ ID NO: 1 has been deleted. Accordingly 9-39 Exendin4 represents a N-terminally truncated Exendin4 peptide wherein the first 8 amino acids have been deleted. In addition a reference to a position greater than 39 (native Exendin4 only has 39 amino acids) is intended to refer to amino acid position in an analog having a C-terminus amino acid extension after the corresponding position 39 of SEQ ID NO: 1.

As used herein the general term “polyethylene glycol chain” or “PEG chain”, refers to mixtures of condensation polymers of ethylene oxide and water, in a branched or straight chain, represented by the general formula H(OCH₂CH₂)ₙOH, wherein n is at least 2. “Polyethylene glycol chain” or “PEG chain” is used in combination with a numeric suffix to indicate the approximate average molecular weight thereof. For example, PEG-5,000 refers to polyethylene glycol chain having a total molecular weight average of about 5,000 Daltons.

As used herein the term “pegylated” and like terms refers to a compound that has been modified from its native state by linking a polyethylene glycol chain to the compound. A “pegylated polypeptide” is a polypeptide that has a PEG chain covalently bound to the polypeptide.

As used herein the term “miniPEG” or “OEG” defines a functionalized polyethylene compound comprising the structure:

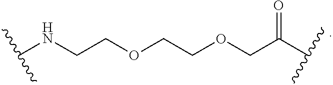 [SMILES: *NCCOCCOCC(*)=O]

As used herein a “linker” or “spacer” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties, and enzyme-cleavable groups.

As used herein a “dimer” is a complex comprising two subunits covalently bound to one another via a linker. The term dimer, when used absent any qualifying language, encompasses both homodimers and heterodimers. A homodimer comprises two identical subunits, whereas a heterodimer comprises two subunits that differ, although the two subunits are substantially similar with one another.

As used herein the term C16-C20 fatty acid designates the structure: —CO(CH₂)₁₄₋₂₀CH₃ and the term C16-C20 diacid designates the structure: —CO(CH₂)₁₄₋₂₀COOH, wherein the prefix “C16-C20” designates the variable total number of carbons in the compounds encompassed by the designation. For example, a C18 diacid represents the structure: —CO(CH₂)₁₆COOH. As used herein a generic reference to an acylated amino acid encompasses both an amino acid having its side chain acylated with a fatty acid and an amino acid having its side chain acylated with a diacid.

Physiological conditions as disclosed herein are intended to include a temperature of about 35 to 40° C. and a pH of about 7.0 to about 7.4, and more typically include a pH of 7.2 to 7.4 and a temperature of 36 to 38° C. Since physiological pH and temperature are tightly regulated in humans within a highly defined range, the speed of conversion from dipeptide/drug complex (prodrug) to drug will exhibit high intra and interpatient reproducibility.

The term “C₁-Cₙ alkyl” wherein n can be from 1 through 6, as used herein, represents a branched or linear alkyl group having from one to the specified number of carbon atoms. Typical C₁-C₆ alkyl groups include, but are not limited to, methyl, ethyl, n-propyl, iso-propyl, butyl, iso-Butyl, sec-butyl, tert-butyl, pentyl, hexyl and the like.

The terms “C₂-Cₙ alkenyl” wherein n can be from 2 through 6, as used herein, represents an olefinically unsaturated branched or linear group having from 2 to the specified number of carbon atoms and at least one double bond. Examples of such groups include, but are not limited to, 1-propenyl, 2-propenyl (—CH₂—CH═CH₂), 1,3-butadienyl, (—CH═CHCH═CH₂), 1-butenyl (—CH═CHCH₂CH₃), hexenyl, pentenyl, and the like.

The term “C₂ -Cₙ alkynyl” wherein n can be from 2 to 6, refers to an unsaturated branched or linear group having from 2 to n carbon atoms and at least one triple bond. Examples of such groups include, but are not limited to, 1-propynyl, 2-propynyl, 1-butynyl, 2-butynyl, 1-pentynyl, and the like.

As used herein the term “aryl” refers to a mono- or bicyclic carbocyclic ring system having one or two aromatic rings including, but not limited to, phenyl, naphthyl, tetrahydronaphthyl, indanyl, indenyl, and the like. The size of the aryl ring and the presence of substituents or linking groups are indicated by designating the number of carbons present. For example, the term “(C₁-C₃ alkyl)(C₆-C₁₀ aryl)” refers to a 5 to 10 membered aryl that is attached to a parent moiety via a one to three membered alkyl chain.

The term “heteroaryl” as used herein refers to a mono- or bi-cyclic ring system containing one or two aromatic rings and containing at least one nitrogen, oxygen, or sulfur atom in an aromatic ring. The size of the heteroaryl ring and the presence of substituents or linking groups are indicated by designating the number of carbons present. For example, the term “(C₁-Cₙ alkyl)(C₅-C₆ heteroaryl)” refers to a 5 or 6 membered heteroaryl that is attached to a parent moiety via a one to “n” membered alkyl chain

As used herein, the term “halo” refers to one or more members of the group consisting of fluorine, chlorine, bromine, and iodine.

As used herein the term “patient” without further designation is intended to encompass any warm blooded vertebrate domesticated animal (including for example, but not limited to livestock, horses, cats, dogs and other pets) and humans and is not limited to individuals under the direct care of a physician.

The term “isolated” as used herein means having been removed from its natural environment.

The term “purified,” as used herein relates to the isolation of a molecule or compound in a form that is substantially free of contaminants normally associated with the molecule or compound in a native or natural environment and means having been increased in purity as a result of being separated from other components of the original composition. The term “purified peptide” is used herein to describe a peptide which has been separated from other compounds including, but not limited to nucleic acid molecules, lipids and carbohydrates.

As used herein, the term “peptide” encompasses a sequence of 2 or more amino acids and typically less than 50 amino acids, wherein the amino acids are naturally occurring or coded or non-naturally occurring or non-coded amino acids. Non-naturally occurring amino acids refer to amino acids that do not naturally occur in vivo but which, nevertheless, can be incorporated into the peptide structures described herein. “Non-coded” as used herein refer to an amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

As used herein, “partly non-peptidic” refers to a molecule wherein a portion of the molecule is a chemical compound or substituent that has biological activity and that does not comprise a sequence of amino acids.

A “peptidomimetic” refers to a chemical compound having a structure that is different from the general structure of an existing peptide, but that functions in a manner similar to the existing peptide, e.g., by mimicking the biological activity of that peptide. Peptidomimetics typically comprise naturally occurring amino acids and/or unnatural amino acids, but can also comprise modifications to the peptide backbone. For example, a peptidomimetic may include a sequence of naturally-occurring amino acids with the insertion or substitution of a non-peptide moiety, e.g. a retroinverso fragment, or incorporation of non-peptide bonds such as an azapeptide bond (CO substituted by NH) or pseudo-peptide bond (e.g. NH substituted with CH₂), or an ester bond (e.g., depsipeptides, wherein one or more of the amide (—CONHR—) bonds are replaced by ester (COOR) bonds). Alternatively the peptidomimetic may be devoid of any naturally-occurring amino acids.

As used herein the term “charged amino acid” or “charged residue” refers to an amino acid that comprises a side chain that is negatively charged (i.e., de-protonated) or positively charged (i.e., protonated) in aqueous solution at physiological pH. For example, negatively charged amino acids include aspartic acid, glutamic acid, cysteic acid, homocysteic acid, and homoglutamic acid, whereas positively charged amino acids include arginine, lysine and histidine. Charged amino acids include the charged amino acids among the 20 amino acids commonly found in human proteins, as well as atypical or non-naturally occurring amino acids.

As used herein the term “acidic amino acid” refers to an amino acid that comprises a second acidic moiety (other than the alpha carboxylic acid of the amino acid), including for example, a side chain carboxylic acid or sulfonic acid group.

As used herein, the term “prodrug” is defined as any compound that undergoes chemical modification before exhibiting its full pharmacological effects.

As used herein, a “dipeptide” is the result of the linkage of an α-amino acid or α-hydroxyl acid to another amino acid, through a peptide bond.

As used herein the term “chemical cleavage” absent any further designation encompasses a non-enzymatic reaction that results in the breakage of a covalent chemical bond.

As used herein the term “atypical hypoglycemia” defines a condition of hypoglycemia occurring in a patient independent of exogenous insulin administration.

### Abbreviations

Lower case k=D-isomer of lysine
Upper case K=L-isomer of lysine
γE=L-isomer of gamma, glutamic acid
(miniPEG)₂=COCH₂OCH₂CH₂OCH₂CH₂NH
COC₁₆H₃₂CO₂H=(C18 diacid)
(N-Me)G=sarcosine

### Embodiments

In accordance with one embodiment of the present disclosure, compositions and methods are provided for treating patients suffering from a hypoglycemic condition that results independently of exogenous insulin administration (i.e., atypical hypoglycemia). In accordance with the present disclosure a composition comprising a GLP-1 antagonist is administered to a patient suffering from atypical hypoglycemia in an amount sufficient to increase blood glucose levels and/or alleviate associated acute symptoms and chronic outcomes associated with hypoglycemia. In one embodiment the patient experiencing atypical hypoglycemia also has a condition of hyperinsulinemia. In one embodiment the hyperinsulinemic condition occurs after the patient has received bariatric surgery.

In accordance with one embodiment a GLP-1 receptor antagonist peptide is provided that is an analog of the peptide DVSSYLEEQAVREFIAWLVKGGPSSGAPPPSK (SEQ ID NO: 3), wherein the GLP-1 receptor antagonist differs from SEQ ID NO: 3 by 1, 2, 3, 4, 5, 6, 7, 8, 9 or 10 amino acid modifications, wherein the modifications are selected from amino acid substitutions, additions or modifications to the amino acid structure, including but not limited to acylation and/or amidation of the C terminal amino acid. In one embodiment the amino acid modification are amino acid substitutions at one or more of positions 12, 16, 18, 19, 24, 26, 27 and 28 of the peptide and/or substitution with the d-isomer at one or more of positions 15, 16, 17, 19, 20, 21 and 23 (numbering relative to native Exendin4 (SEQ ID NO: 1). In one embodiment the analog of SEQ ID NO: 3 is further modified by the acylation of the C-terminal amino acid side chain with a C16-C18 fatty acid or diacid, optionally via a spacer.

In accordance with one embodiment a GLP-1 receptor antagonist is provided comprising the amino acid sequence of DVSRYLEEQAVREFlEWLVRGGPSSGAPPPSK (SEQ ID NO: 4), or an amino acid sequence that differs from SEQ ID NO: 4 by 1, 2, 3, 4 or 5 amino acid substitutions while retaining GLP-1 receptor antagonist activity, with the proviso that the GLP-1 antagonist peptide does not comprise the sequence of SEQ ID NO: 3. In one embodiment a peptide is provided that differs from the peptide of SEQ ID NO: 4 by one or more of the following:
i) substitution of 1, 2 or 3 amino acids at any of positions 7, 10, 11, 13 or 16 with Trp or dTrp;
ii) substitution of 1, 2 or 3 amino acids at any of positions 15, 16 or 23 with the corresponding amino acid in the D conformation;
iii) substitution of 1, 2 or 3 amino acids at any of positions 16, 18, 19, 24, 26, or 28 with Aib;
iv) substitution at position 12 with an acylated amino acid, optionally an acylated lysine;
v) substitution at position 16 with dGlu, Asp, homoglutamic acid or homocysteic acid;
vi) substitution at position 19 with cyclopropane, cyclopentane, cyclohexane or phenyl glycine;
vii) substitution at position 20 with dArg, homolysine or citrulline;
viii) substitution of the native C-terminal carboxyl group with an amide;
ix) addition of a N-terminal extension of 1 to 3 amino acids, optionally wherein one of the N-terminal extension amino acids is acylated;
x) substitution of the C-terminal amino acid with an acylated amino acid;
xi) any combination of i) through x).

In accordance with one embodiment a GLP-1 receptor antagonist is provided comprising the amino acid sequence of R₁₀-DVX₁₁X₁₂YLX₁₅X₁₆QAX₁₉X₂₀EFX₂₃EWLVRGGPSSGAPPPSX₄₀-R₂₀ SEQ ID NO: 23), wherein
R₁₀ is NH₂ or an N-terminal extension of 1, 2 or 3 amino acids, wherein one of the amino acids of the N-terminal extension is acylated with a C16-C18 fatty acid or diacid, optionally via a spacer, optionally wherein R₁₀ is a dipeptide of the structure: X₇X₈-, wherein X₇ is an acylated amino acid, optionally acylated Lys or acylated dLys, and X₈ is Gly or a C₁-C₄ N-alkylated Gly;
X₁₁ is Trp, dTrp or Ser;
X₁₂ is Arg or an acylated amino acid, optionally acylated Lys;
X₁₅ is Glu or dGlu;
X₁₆ is Glu, dGlu, Asp, homoglutamic acid or homocysteic acid;
X₁₉ is Val, cyclopropane, cyclopentane, cyclohexane or phenyl glycine;
X₂₀ is Arg, homolysine or citrulline;
X₂₃ is Ile, or dIle;
X₄₀ is an acylated amino acid, optionally an acylated Lys; and
R₂₀ is COOH or CONH₂-, optionally wherein 1, 2 or 3 amino acids selected from positions 7, 10, 13, or 16 are substituted with Trp or dTrp, with an optional proviso that positions 10 and 11 are not both Trp or dTrp; with a further optional proviso that that R₁₀ and X₁₂ cannot both comprise an acylated amino acid; optionally wherein 1, 2 or 3 amino acids at any of positions 16, 18, 19, 24, 26, or 28 are substituted with Aib.

In one embodiment only one of positions 7, 12 and 40 of the peptide of SEQ ID NO: 23 comprises an acylated amino acid. In one embodiment two of positions 7, 12 and 40 of the peptide of SEQ ID NO: 23 comprises an acylated amino acid. In one embodiment the acylated amino acids at position 7, 12 and 40 are independently an amino acid comprising a structure of Formula I (optionally, Lys), or Formula II (optionally, Ser), wherein each of Formula I, and II is:

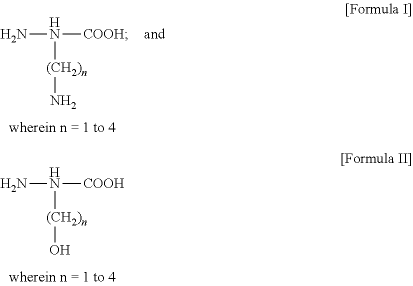

In one embodiment a peptide of SEQ ID NO: 23 is provided wherein R₁₀ is X₇X₈-, wherein X₇ is an acylated Lys or acylated dLys, X₁₂ is Arg and X₄₀ is an acylated Lys. In one embodiment a peptide of SEQ ID NO: 23 is provided wherein R₁₀ is NH₂-, X₁₂ is an acylated Lys and X₄₀ is an acylated Lys. In one embodiment a peptide of SEQ ID NO: 23 is provided wherein R₁₀ is NH₂-, X₁₂ is Arg and X₄₀ is an acylated Lys. In one embodiment the acylated amino acids of the peptide of SEQ ID NO: 23 are acylated Lys residues, optionally wherein the acylated Lys residues are independently acylated with a C14-C24 fatty acid or fatty diacid, or a C16-C18 fatty acid or fatty diacid, optionally wherein the fatty acid or fatty diacid is linked to the side chain of the Lys residue via any of the spacer molecules disclosed herein.

In accordance with one embodiment a GLP-1 receptor antagonist is provided comprising the amino acid sequence of

SEQ ID NO: 24)
R₁₀-DVX₁₁X₁₂YLEX₁₆QAVREFIEWLVRGGPSSGAPPPSX₄₀-R₂₀,

wherein

R₁₀ is NH₂ or a dipeptide of the structure: X₇X₈-, wherein X₇ is an acylated amino acid, optionally acylated Lys or acylated dLys, and X₈ is Gly or a C₁-C₄ N-alkylated Gly;

X₁₁ is Trp, dTrp or Ser;
X₁₂ is Arg or an acylated amino acid, optionally acylated Lys;
X₁₆ is Glu or Asp;
X₄₀ is an acylated amino acid, optionally an acylated Lys; and
R₂₀ is COOH or CONH₂-, optionally wherein 1, 2 or 3 amino acids selected from positions 16, 17, 18, 20 or 21 are substituted with the corresponding amino acid in the D conformation, and/or 1, 2 or 3 amino acids at any of positions 16, 18, 19, 24, 26, or 28 are substituted with Aib.

In accordance with one embodiment a GLP-1 receptor antagonist is provided comprising the amino acid sequence of

SEQ ID NO: 24)
R₁₀-DVX₁₁X₁₂YLEX₁₆QAVREFIEWLVRGGPSSGAPPPSX₄₀-R₂₀,

wherein
R₁₀ is a dipeptide of the structure: X₇X₈-, wherein X₇ is an acylated Lys or acylated dLys, and X₈ is Gly or Sarcosine;
X₁₁ is Trp or dTrp;
X₁₂ is Arg;
X₁₆ is Glu;
X₄₀ is an acylated Lys; and
R₂₀ is COOH or CONH₂-, wherein said acylated Lys residues of the peptide are independently acylated with a C16-C18 fatty acid or fatty diacid optionally via a spacer as disclosed herein, optionally wherein 1, 2 or 3 amino acids at any of positions 16, 18, 19, 24, 26, or 28 are substituted with Aib.

In accordance with one embodiment a GLP-1 receptor antagonist is provided comprising the amino acid sequence of

SEQ ID NO: 97)
R₁₀-DVX₁₁X₁₂YLEX₁₆QAVREFIEWLVRGGPSSGAPPPS-R₂₀,

wherein
R₁₀ is a dipeptide of the structure: X₇X₈-, wherein X₇ is an acylated Lys or acylated dLys, and X₈ is Gly or Sarcosine;
X₁₁ is Ser, Trp or dTrp;
X₁₂ is Arg;
X₁₆ is Glu;
and
R₂₀ is COOH or CONH₂-, wherein said acylated Lys residues is acylated with a C16-C18 fatty acid or fatty diacid optionally via a spacer as disclosed herein, optionally wherein 1, 2 or 3 amino acids at any of positions 16, 18, 19, 24, 26, or 28 are substituted with Aib.

In one embodiment the GLP-1 antagonist peptide comprises an amino acid sequence of DVX₁₁RYLQX₁₅X₁₆AVREFX₂₃EWLVRGGPSSGAPPPSX₄₀-R₂₀ (SEQ ID NO: 25) wherein
X₁₁ is Trp, dTrp or Ser;
X₁₅ is Glu or dGlu;
X₁₆ is Glu or dGlu;
X₂₃ is Ile, or dIle;
X₄₀ is an acylated amino acid, optionally a Lys acylated with a C16-C18 fatty acid or diacid, optionally via a spacer; and
R₂₀ is COOH or CONH₂-. Optionally, the GLP-1 antagonist of SEQ ID NO: 25 is further modified by one or more Aib substitutions at any of positions 16, 18, 19, 24, 26, or 28 relative to the numbering of the native Exendin4 sequence of SEQ ID NO: 1, or optionally a substitution of an acylated Lys at position 12. In one embodiment a peptide of SEQ ID NO: 25 is provided further comprising a substitution of an acylated Lys at position 12 and an optional substitution of Aib at position 27.

In one embodiment the GLP-1 antagonist peptide comprises an amino acid sequence of
DVX₁₁RYLEEQAVREFIEWLVRGGPSSGAPPPSX₄₀R₂ (SEQ ID NO: 6) or
X₇X₈DVX₁₁RYLEEQAVREFIEWLVRGGPSSGAPPPSX₄₀R₂₀ (SEQ ID NO:26) wherein X₇ is an acylated Lys or acylated dLys, and X₈ is Gly or Sarcosine, X₁₁ is Trp or dTrp, X₄₀ is an acylated amino acid, optionally an acylated Lys, wherein the acylated Lys residues comprise a C16-C18 fatty acid or diacid covalently linked to the Lys side chain, optionally via a spacer, and R₂₀ is COOH or CONH₂-. Optionally, the peptides of SEQ ID NO: 6 and SEQ ID NO: 26 can be further modified with an Aib substitution at any one of positions at any of positions 16, 18, 19, 24, 26 or 28 based on the numbering of native Exendin4 (SEQ ID NO: 1) or the peptide of SEQ ID NO: 6 is optionally substituted with an acylated Lys at position 12.

In one embodiment the GLP-1 antagonist peptide comprises an amino acid sequence of DVX₁₁RYLEEQAVREFIEWLVRGGPSSGAPPPSX₄₀R₂₀ (SEQ ID NO: 6 or DVWRYLEEQAVREFIEWLVRGGPSSGAPPPSX₄₀R₂₀ (SEQ ID NO: 7) or an amino acid that differs from SEQ ID NO: 6 or SEQ ID NO: 7 by 1 or 2 amino acid substitutions, wherein X₁₁ is Trp or dTrp, X₄₀ is an amino acid having an acyl group of sufficient sized to bind serum albumin with high affinity linked to the side chain of the amino acid, optionally where the acyl group is linked via a spacer, and R₂₀ is COOH or CONH₂ optionally with an Aib substitution at any one of positions 16, 18, 19, 24, 26 or 28 relative the numbering of native Exendin4 (SEQ ID NO: 1) and optionally wherein the carboxy group of the C-terminal amino acid is substituted with an amide (i.e., R₂₀ is CONH₂). In one embodiment X₄₀ is an amino acid comprising a structure of Formula I (optionally, Lys), Formula II (optionally, Ser), or Formula III (optionally, Cys), wherein each of Formulae I, II, and III, is:

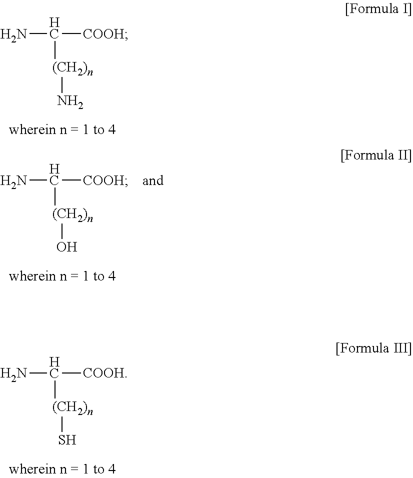

In one embodiment the GLP-1 antagonist peptide comprises an amino acid sequence of

(SEQ ID NO: 9)
DVX₁₁RYLEEQAVREFIEWLVRGGPSSGAPPPSX₄₀-COOH
or
(SEQ ID NO: 10)
DVX₁₁RYLEEQAVREFIEWLVRGGPSSGAPPPSX₄₀-NH₂,

10), wherein X₁₁ is Trp or dTrp, and X₄₀ is an amino acid having an acyl group linked to the side chain of the amino acid, optionally via a spacer. In one embodiment X₄₀ is an acylated Lys.

In accordance with one embodiment a GLP-1 receptor antagonist peptide is provided having the amino acid sequence of

(SEQ ID NO: 8)
DVWX₁₂YLEEQAVREFIEWLVRGGPSSGAPPPSX₄₀-NH₂,

wherein
X₁₂ is an acylated Lys; and
X₄₀ is an acylated Lys having an amide substituting for the C-terminal carboxylic acid, wherein the acyl group of the acylated Lys is a C16-C18 acid or diacid, optionally linked via a spacer to the Lys side chain.

In yet another aspect, the amino acid at position 1 of any of the GLP-1 receptor antagonists disclosed herein is modified to inhibit protease degradation of the peptide. In one embodiment, inhibition of proteases is accomplished by:
i) acylating the side chain of the N-terminal amino acid;
ii) substituting the N-terminal amino acid with its D-stereoisomer;
iii) modifying the N-terminal alpha amine, including for example, covalently linking an acetyl group to the alpha amine, or removing the alpha amine group; or
iv) any combination of i)-iii).

In one embodiment a peptide comprising the sequence of DVX₁₁RYLEEQAVREFIEWLVRGGPSSGAPPPSX₄₀R₂ (SEQ ID NO: 6) is provided having up to 3 amino acid modifications relative to SEQ ID NO: 6, wherein XII is Trp or dTrp, X₄₀ is an acylated amino acid, and R₂₀ is COOH or CONH₂-, wherein the peptide exhibits antagonist activity at the human GLP-1.

In one embodiment the GLP-1 receptor antagonist comprises a peptide selected from the group consisting of

(SEQ ID NO: 27)
DV(dW)RYLEEQAVREFIEWLVRGGPSSGAPPPSX₄₀ R₂₀,
(SEQ ID NO: 28)
DVWRYLEEQAVREFIEWLVRGGPSSGAPPPSX₄₀ R₂₀,
(SEQ ID NO: 29)
DV(dW)RYLE(Aib)QAVREFIEWLVRGGPSSGAPPPSX₄₀ R₂₀,
(SEQ ID NO: 30)
DVWRYLE(Aib)QAVREFIEWLVRGGPSSGAPPPSX₄₀ R₂₀,
(SEQ ID NO: 31)
DV(dW)RYLEEQ(Aib)VREFIEWLVRGGPSSGAPPPSX₄₀ R₂₀,
(SEQ ID NO: 32)
DVWRYLEEQ(Aib)VREFIEWLVRGGPSSGAPPPSX₄₀ R₂₀,
(SEQ ID NO: 33)
DV(dW)RYLEEQA(Aib)REFIEWLVRGGPSSGAPPPSX₄₀ R₂₀,
(SEQ ID NO: 34)
DVWRYLEEQA(Aib)REFIEWLVRGGPSSGAPPPSX₄₀ R₂₀,
(SEQ ID NO: 35)
DV(dW)RYLEEQAVREFI(Aib)WLVRGGPSSGAPPPSX₄₀ R₂₀,
(SEQ ID NO: 36)
DVWRYLEEQAVREFI(Aib)WLVRGGPSSGAPPPSX₄₀ R₂₀,
(SEQ ID NO: 37)
DV(dW)RYLEEQAVREFIEW(Aib)VRGGPSSGAPPPSX₄₀ R₂₀,
(SEQ ID NO: 38)
DVWRYLEEQAVREFIEW(Aib)VRGGPSSGAPPPSX₄₀ R₂₀,
(SEQ ID NO: 39)
DV(dW)RYLEEQAVREFIEWLV(Aib)GGPSSGAPPPSX₄₀ R₂₀,
(SEQ ID NO: 40)
DVWRYLEEQAVREFIEWLV(Aib)GGPSSGAPPPSX₄₀ R₂₀,
(SEQ ID NO: 41)
DV(dW)RYLEEQAV(dR)EFIEWLVRGGPSSGAPPPSX₄₀ R₂₀,
(SEQ ID NO: 42)
DVWRYLEEQAV(dR)EFIEWLVRGGPSSGAPPPSX₄₀ R₂₀,

wherein X₄₀ is an acylated amino acid, optionally a Lys acylated with a C16-C18 fatty acid or diacid, and R₂₀ is COOH or CONH₂-, wherein the peptide exhibits antagonist activity at the human GLP-1.

Additional exemplified species of the present disclosure include those listed in Table 1:

TABLE 1

| SEQ | | |
|---|---|---|
| ID | Description/cmpd | |
| NO. | registry | Sequence |
| 1 | Exendin-4 | HGEGTSDVSKQMEEEAVRLFIEWLKNGGPSSGAPPPS |
| 2 | Ex-4 (9-39)a | DVSKQMEEEAVRLFIEWLKNGGPSSGAPPPS |
| 3 | Jant4 9-40am; | DVSSYLEEQAVREFIAWLVKGGPSSGAPPPSK[C16]-OH |
| | K40[C16] | |
| 4 | | DVSRYLEEQAVREFIEWLVRGGPSSGAPPPSK |
| 5 | | DX₁₀X₁₁RYLX₁₅X₁₆QAVREFX₂₃EWLVRGGPSSGAPPPSX₄₀ |
| 6 | | DVX₁₁RYLEEQAVREFIEWLVRGGPSSGAPPPSX₄₀ |
| 7 | | DVWRYLEEQAVREFIEWLVRGGPSSGAPPPSX ₄₀ |
| 8 | | DVWX₁₂YLEEQAVREFIEWLVRGGPSSGAPPPSX₄₀-NH₂) |
| 9 | | DVX₁₁RYLEEQAVREFIEWLVRGGPSSGAPPPSX₄₀-COOH |
| 10 | | DVX₁₁RYLEEQAVREFIEWLVRGGPSSGAPPPSX₄₀-NH₂ |
| 11 | | DVX₁₁RYLX₁₅X₁₆QAVREFX₂₃EWLVRGGPSSGAPPPSK |
| 12 | | DVSSYLEEQAVREFIAWLVKGGPSSGAPPPSX₄₀ |
| 13 | | DVSRYLEEQAVREFIEWLVRGGPSSGAPPPSX₄₀ |
| 14 | X₇X₈ = dK(mPeg- | X₇X₈DVSRYLEEQAVREFIEWLVRGGPSSGAPPPSX₄₀ |
| | γE-DiAcidC18)(N- | |
| | Me-Gly) | |
| 15 | X₇X₈ = dK(mPeg- | X₇X₈DVSRYLEEQAVREFIEWLVRGGPSSGAPPPSX₄₀ |
| | γE-DiAcidC18)(N- | |
| | iPr-Gly | |
| 16 | 9-40AC; Jant4 | DVSRYLEEQAVREFIEWLVRGGPSSGAPPPSK[miniPEG- |
| | (R12, E24, R28) | γE-C16]-OH |
| | K40[mPEG-gE- | |
| | C16] | |
| 17 | 9-40AC, dK7(mPeg- | dK(mPeg-γE-DiAcidC18) |
| | γE(DiAcid C18))G8 | GDVSRYLEEQAVREFIEWLVRGGPSSGAPPPS |
| | R12, E24, R28, | K[miniPEG-gE-C16]-OH |
| | K40(mPeg-γE(C16)) | |
| | (Pro-drug) | |
| 18 | 9-40AC, dK7(mPeg- | dK(mPeg-γE-DiAcidC18)(N-iPr- |
| | γE(DiAcid C18)) | Gly)DVSRYLEEQAVREFIEWLVRGGPSSGAPPPSK[miniP |
| | (N-iPr-Gly8) | EG-gE-C16]-OH |
| | R12, E24, R28, | |
| | K40(mpeg-γE(C16)) | |
| | (Pro-drug) | |
| 19 | 9-40AC, dK7(mPeg- | dK(mPeg-γE-DiAcidC18)(N-Me- |
| | γE(DiAcid C18)) | Gly8)DVSRYLEEQAVREFIEWLVRGGPSSGAPPPSK[miniP |
| | (N-Me-Gly8) | EG-gE-C16]-OH |
| | R12, E24, R28, | |
| | K40(mpeg-γE(C16)) | |
| | (Pro-drug) | |
| 20 | Jant9-40am, | dK7(C18diacid-gE-mPeg-K(mPeg-gE- |
| | dK7(C18diacid-gE- | C18diacid)GDVSRYLEEQAVREFIEWLVRGGPSSGAPPPSK |
| | mPeg-K(mPeg-gE- | [miniPEG-gE-C18diacid)-NH2 |
| | C18discid), G8, | |
| | K40(mpeg-gE-C18 | |
| | diacid) | |
| 21 | Jant9-40am, | dK7(C18diacid-gE-mPeg-K(mPeg-gE- |
| | dK7(C18diacid-gE- | C18diacid)GDVSRYLEEQAVREFIEWLVRGGPSSGAPPPSK |
| | mPeg-K(mPeg-gE- | [miniPEG-gE-C16)-NH2 |
| | C18discid), G8, | |
| | K40(mPeg-gE-C16) | |
| 22 | Jant9-40am, | dK7(C18diacid-gE-mPeg-K(mPeg-gE-C18diacid)- |
| | dK7(C18diacid-gE- | N-MeGly-DVSRYLEEQAVREFIEWLVRGGPSSGAPPPSK |
| | mPeg-K(mPeg-gE- | [miniPEG-gE-C16)-NH2 |
| | C18discid), N- | |
| | meGly, K40(mPeg- | |
| | gE-C16) | |
| 23 | | DVX₁₁X₁₂YLX₁₅X₁₆QAX₁₉X₂₀EFX₂₃EWLVRGGPSSGAPPPSX₄₀ |
| 24 | | DVX₁₁X₁₂YLEX₁₆QAVREFIEWLVRGGPSSGAPPPSX₄₀ |
| 25 | | DVX₁₁RYLQX₁₅X₁₆AVREFX₂₃EWLVRGGPSSGAPPPSX₄₀ |
| 26 | | X₇X₈DVX₁₁RYLEEQAVREFIEWLVRGGPSSGAPPPSX₄₀R₂₀ |
| 27 | | DV(dW)RYLEEQAVREFIEWLVRGGPSSGAPPPSX₄₀ R₂₀ |
| 28 | | DVWRYLEEQAVREFIEWLVRGGPSSGAPPPSX₄₀ R₂₀ |
| 29 | | DV(dW)RYLE(Aib)QAVREFIEWLVRGGPSSGAPPPSX₄₀ R₂₀ |
| 30 | | DVWRYLE(Aib)QAVREFIEWLVRGGPSSGAPPPSX₄₀ R₂₀ |
| 31 | | DV(dW)RYLEEQ(Aib)VREFIEWLVRGGPSSGAPPPSX₄₀ R₂₀ |
| 32 | | DVWRYLEEQ(Aib)VREFIEWLVRGGPSSGAPPPSX₄₀ R₂₀ |
| 33 | | DV(dW)RYLEEQA(Aib)REFIEWLVRGGPSSGAPPPSX₄₀ R₂₀ |
| 34 | | DVWRYLEEQA(Aib)REFIEWLVRGGPSSGAPPPSX₄₀ R₂₀ |
| 35 | | DV(dW)RYLEEQAVREFI(Aib)WLVRGGPSSGAPPPSX₄₀ R₂₀ |
| 36 | | DVWRYLEEQAVREFI(Aib)WLVRGGPSSGAPPPSX₄₀ R₂₀ |
| 37 | | DV(dW)RYLEEQAVREFIEW(Aib)VRGGPSSGAPPPSX₄₀ R₂₀ |
| 38 | | DVWRYLEEQAVREFIEW(Aib)VRGGPSSGAPPPSX₄₀ R₂₀ |
| 39 | | DV(dW)RYLEEQAVREFIEWLV(Aib)GGPSSGAPPPSX₄₀ R₂₀ |
| 40 | | DVWRYLEEQAVREFIEWLV(Aib)GGPSSGAPPPSX₄₀ R₂₀ |
| 41 | | DV(dW)RYLEEQAV(dR)EFIEWLVRGGPSSGAPPPSX₄₀ R₂₀ |
| 42 | | DVWRYLEEQAV(dR)EFIEWLVRGGPSSGAPPPSX₄₀ R₂₀ |
| 43 | 9-40am; Jant4 | DVSSYLEEQAVREFIAWLK(Aib)GGPSSGAPPPSK[C16]- |
| | (K27, Aib28) | NH2 |
| | K40[C16] | |
| 44 | 9-40am; Jant4 | DVSSYLEEQAVREFIAW(Aib)VKGGPSSGAPPPSK[C16]- |
| | (Aib26) K40[C16] | NH2 |
| 45 | 9-40am; Jant4 | DVSSYLEEQA(Aib)REFIAWLVKGGPSSGAPPPSK[C16]- |
| | (Aib19) K40[C16] | NH2 |
| 46 | 9-40am; Jant4 | DVSSYLEEQ(Aib)VREFIAWLVKGGPSSGAPPPSK[C16]- |
| | (Aib18) K40[C16] | NH2 |
| 47 | 9-40am; Jant4 | DVSSYLEEQAVREFIAWLVKGGPSSGAPPPSK[C16]-NH2 |
| | K40[C16] | |
| 48 | 9-40am; Jant4 | DVSSYLEEQAVREFIAWLVKGGPSSGAPPPSK |
| | K40[(mPEG)2-gE- | [(miniPEG)2-gE-C16]-NH2 |
| | C16] | |
| 49 | 9-40am; Jant4 | DVSSYL(dE)EQAVREFIAWLVKGGPSSGAPPPSK[C16]- |
| | (dE15) K40[C16] | NH2 |
| 50 | 9-40am; Jant4 | DVSSYLEEQA(dV)REFIAWLVKGGPSSGAPPPSK[C16]- |
| | (dV19) K40[C16] | NH2 |
| 51 | 9-40am; Jant4 | DVSSYLEEQAVREF(dI)AWLVKGGPSSGAPPPSK[C16]- |
| | (dI23) K40[C16] | NH2 |
| 52 | 9-40am; Jant4 | DVSSYLEEQAVREFIAWLVKGGPSSGAPPPSK[gE-C16]- |
| | K40[gE-C16] | NH2 |
| 53 | 9-40am; Jant4 | DVSSYLEEQAVREFIAWLVKGGPSSGAPPPSK[gE2-C16]- |
| | K40[gE2-C16] | NH2 |
| 54 | 9-40am; Jant4 | DVSSYLEEQAVREFIAWLVKGGPSSGAPPPSK[gE4-C16]- |
| | K40[gE4-C16] | NH2 |
| 55 | 9-40am; Jant4 | DVSSYLEEQAVREFIAWLVKGGPSSGAPPPSK[gE6-C16]- |
| | K40[gE6-C16] | NH2 |
| 56 | 9-40am; Jant4 | DVSSYLEEQAVREFIAWLVKGGPSSGAPPPSK[miniPEG- |
| | K40[mPEG-gE- | gE-C16]-NH2 |
| | C16] | |
| 57 | 9-40am; Jant4 | DVSSYLEEQAVREFIAWLVKGGPSSGAPPPSK[gE- |
| | K40[EP2E-C16] | (miniPEG)2-gE-C16]-NH2 |
| 58 | 9-40am; Jant4 | DVSSYLEEQAVREFIAWLVKGGPSSGAPPPSK[(miniPEG)2 |
| | K40(P2E2-C16) | -gE2-C16]-NH2 |
| 59 | 9-40am; Jant4 | DVSSYLEEQAVREFIAWLVKGGPSSGAPPPSK[miniPEG- |
| | K40(PEPE-C16) | gE-miniPEG-gE-C16]-NH2 |
| 60 | 9-40am; Jant4 | DVSSYLEEQAVREFIAWLVKGGPSSGAPPPSK[miniPEG- |
| | K40[PE2P-C16] | gE2-miniPEG-C16]-NH2 |
| 61 | 9-40am; Jant4 | DVSSYLEEQAVREFIAWLVKGGPSSGAPPPSK[gE2- |
| | K40[E2P2-C16] | (miniPEG)2-C16]-NH2 |
| 62 | 9-40am; Jant4 | DVSSYLEEQAVREFIAWLVKGGPSSGAPPPSK[gE- |
| | K40[EPEP-C16] | miniPEG-gE-miniPEG-C16]-NH2 |
| 63 | 9-40am;Jant4 | DVSSYLEEQAVREFIAWLVKGGPSSGAPPPSK[DiAcid |
| | K40[DiAcC18] | C18]-NH2 |
| 64 | 9-40am; Jant4 | DVSSYLEEQAVREFIAWLVKGGPSSGAPPPSK[gE2- |
| | K40[gE2-DiAcC18] | DiAcidC18]-NH2 |
| 65 | 9-40am; Jant4 | DVSSYLEEQAVREFIAWLVKGGPSSGAPPPSK[gE4- |
| | K40[gE4-DiAcC18] | DiAcidC18]-NH2 |
| 66 | 9-40am; Jant4 | DVSSYLEEQAVREFIAWLVKGGPSSGAPPPSK[gE6- |
| | K40[gE6-DiAcC18] | DiAcidC18]-NH2 |
| 67 | 9-40am; Jant4 | DVSSYLEEQAVREFIAWLVKGGPSSGAPPPSK[(miniPEG)2 |
| | K40[(mPEG)2-gE- | -gE-DiAcidC18]-NH2 |
| | DiAC18] | |
| 68 | 9-40am; Jant4 | DVSSYLEEQAVREFIAWLVKGGPSSGAPPPSK[miniPEG- |
| | K40[mPEG-gE- | gE-DiAcC18]-NH2 |
| | DiAcC18] | |
| 69 | 9-40am; Jant4 (R28) | DVSSYLEEQAVREFIAWLVRGGPSSGAPPPSK[C16]-NH2 |
| | K40[C16] | |
| 70 | 9-40am; Jant4 | DVSSYLEEQAVREFIEWLVRGGPSSGAPPPSK[C16]-NH2 |
| | (E24, R28) | |
| | K40[C16] | |
| 71 | 9-40am; Jant4 | DVSSYLEEEAVREFIAWLVRGGPSSGAPPPSK[C16]-NH2 |
| | (E17, R28) | |
| | K40[C16] | |
| 72 | 9-40am; Jant4 | DVSSYLEEEAVREFIEWLVRGGPSSGAPPPSK[C16]-NH2 |
| | (E17, E24, R28) | |
| | K40[C16] | |
| 73 | 9-40am; Jant4 | DVSRYLEEQAVREFIAWLVRGGPSSGAPPPSK[C16]-NH2 |
| | (R12, R28) | |
| | K40[C16] | |
| 74 | 9-40am; Jant4 | DVSRYLEEQAVREFIEWLVRGGPSSGAPPPSK[C16]-NH2 |
| | (R12, E24, R28) | |
| | K40[C16] | |
| 75 | 9-40am; Jant4 | DVSRYLEEEAVREFIEWLVRGGPSSGAPPPSK[C16]-NH2 |
| | (R12, E17, E24, R28) | |
| | K40[C16] | |
| 76 | Ex9 K40[C16] | DLSKQMEEEAVRLFIEWLKNGGPSSGAPPPSK[C16]-NH2 |
| 77 | 9-40AC; Jant4 | DVSSYLEEQAVREFIAWLVRGGPSSGAPPPSK[miniPEG- |
| | (R28) K40[mPEG- | gE-C16]-OH |
| | gE-C16] | |
| 78 | 9-40AC; Jant4 | DVSSYLEEQAVREFIEWLVRGGPSSGAPPPSK[miniPEG- |
| | (E24, R28) | gE-C16]-OH |
| | K40[mPEG-gE- | |
| | C16] | |
| 79 | 9-40AC; Jant4 | DVSSYLEEQAVREFIEWLRVGGPSSGAPPPSK[miniPEG- |
| | (E24, R27, V28) | gE-C16]-OH |
| | K40[mPEG-gE- | |
| | C16] | |
| 80 | 9-40AC; Jant4 | DVSSYLEEQAVREFIEWLR(Aib)GGPSSGAPPPSK |
| | (E24, R27, Aib28) | [miniPEG-gE-C16]-OH |
| | K40[mPEG-gE- | |
| | C16] | |
| 81 | 9-40am; Jant4 | DVSSYLEEQAVREFIAWL(Aib)KGGPSSGAPPPSK[C16]- |
| | (Aib27) K40[C16] | NH2 |
| 82 | 9-40am; Jant4 (K26, | DVSSYLEEQAVREFIAWKV(Aib)GGPSSGAPPPSK[C16]- |
| | Aib28) K40[C16] | NH2 |
| 83 | 9-40am; Jant4 | DVSSYLEEQAVREFI(Aib)WLVKGGPSSGAPPPSK[C16]- |
| | (Aib24) K40[C16] | NH2 |
| 84 | 9-40am; Jant4 | DVSSYLE(Aib)QAVREFIAWLVKGGPSSGAPPPSK[C16]- |
| | (Aib16) K40[C16] | NH2 |
| 85 | 9-40am; Jant4 | DVSSYLE(dE)QAVREFIAWLVKGGPSSGAPPPSK[C16]- |
| | (dE16) K40[C16] | NH2 |
| 86 | 9-40am; Jant4 | DVSSYLEE(dQ)AVREFIAWLVKGGPSSGAPPPSK[C16]- |
| | (dQ17) K40[C16] | NH2 |
| 87 | 9-40am; Jant4 | DVSSYLEEQAV(dR)EFIAWLVKGGPSSGAPPPSK[C16]- |
| | (dR20) K40[C16] | NH2 |
| 88 | 9-40am; Jant4 | DVSSYLEEQAVR(dE)FIAWLVKGGPSSGAPPPSK[C16]- |
| | (dE21) K40[C16] | NH2 |
| 89 | Ex9, dK7(PEG2- | dK(Peg2-gE(C16)) |
| | γE(C16)), G8 | GDLSKQMEEEAVRLFIEWLKNGGPSSGAPPPS-NH2 |
| 90 | Ex9-40am, K40(C18 | DLSKQMEEEAVRLFIEWLKNGGPSSGAPPPSK(C18 |
| | diacid) | diacid)-NH2 |
| 91 | Ex9-40am, W11, | DLWKQMEEEAVRLFIEWLKNGGPSSGAPPPSK(C18 |
| | K40(C18 diacid) | DiAcid)-NH2 |
| 92 | Ex9-40am, dW11, | DLdWKQMEEEAVRLFIEWLKNGGPSSGAPPPSK(C18 |
| | K40(C18 diacid) | diacid)-NH2 |
| 93 | Ex9-40am, W11, | DLWKQMEEEAVRLFIEWLKNGGPSSGAPPPSK(mPeg- |
| | K40(mPeg-gE-C18 | gE(C18 diacid))-NH2 |
| | diacid) | |
| 94 | Ex9-40am, | DLdWKQMEEEAVRLFIEWLKNGGPSSGAPPPSK(mPeg- |
| | dW11, K40(mPeg- | gE(C18 diacid))-NH2 |
| | gE-C18 diacid) | |
| 95 | 9-40am; Jant4 K40 | DVSSYLEEQAVREFIAWLVKGGPSSGAPPPSK-NH2 |

In one embodiment dimers and multimers comprising two or more GLP-1 receptor antagonist peptides of the present disclosure are prepared including homo- or hetero- multimers or homo- or hetero- dimers. Two or more GLP-1 receptor antagonist peptides can be linked together using standard linking agents and procedures known to those skilled in the art. For example, dimers can be formed between two peptides through the use of bifunctional thiol crosslinkers and bi-functional amine crosslinkers, particularly for GLP-1 receptor antagonist peptides comprising, or substituted with, cysteine, lysine, ornithine, homocysteine or acetyl phenylalanine residues. The dimer can be a homodimer or alternatively can be a heterodimer. In exemplary embodiments, the linker connecting the two (or more) analogs is PEG, e.g., a 5 kDa PEG, 20 kDa PEG. In some embodiments, the linker is a disulfide bond. For example, each monomer of the dimer may comprise a Cys residue (e.g., a terminal or internally positioned Cys) and the sulfur atom of each Cys residue participates in the formation of the disulfide bond. In exemplary aspects, each monomer of the dimer is linked via a thioether bond. In exemplary aspects, an epsilon amine of a Lys residue of one monomer is bonded to a Cys residue, which, in turn, is connected via a chemical moiety to the epsilon amine of a Lys residue of the other monomer.

In some embodiments, the monomers are connected via terminal amino acids (e.g., N-terminal or C-terminal, optionally wherein the amino acid is added to the terminus of a peptide to be dimerized), via internal amino acids, or via a terminal amino acid of at least one monomer and an internal amino acid of at least one other monomer. In some embodiments, the monomers of the multimer are attached together in a “tail-to-tail” orientation in which the C-terminal amino acids of each monomer are attached together. Alternatively, in one embodiment the multimer are attached together in a “head-to-head” orientation in which the N-terminal amino acids of each monomer are attached together.

In one embodiment the C-terminal amino acid of any of the GLP-1 antagonist peptides disclosed herein can be modified to replace the native carboxyl group with an amide. In one embodiment the C-terminal amino acid of any of the GLP-1 antagonist peptides disclosed herein comprises the native amino acid carboxyl group.

### Pharmaceutical Compositions

Pharmaceutical compositions comprising any of the GLP-1 receptor antagonist peptides, dimers, multimers, or conjugates of the present disclosures (or a combination thereof) and a pharmaceutically acceptable carrier, diluent, or excipient are further provided by the present disclosure. The pharmaceutical compositions are preferably sterile and suitable for parenteral administration.

In accordance with one embodiment a pharmaceutical composition is provided comprising any of the novel GLP-1 receptor antagonists disclosed herein, preferably at a purity level of at least 90%, 91%, 92%, 93%, 94%, 95%, 96%, 97%, 98% or 99%, and a pharmaceutically acceptable diluent, carrier or excipient. Such compositions may contain a GLP-1 receptor antagonist as disclosed herein at a concentration of at least 0.1-10 mg/ml, or higher. In one embodiment the pharmaceutical compositions comprise aqueous solutions that are sterilized and optionally stored within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature. In one embodiment the pharmaceutical composition and/or kit comprises any of the GLP-1 receptor antagonists disclosed herein in combination with any existing therapeutics useful for treating hypoglycemia, including but not limited to glucose supplements (eg, dextrose); glucose-elevating agents such as glucagon and glucagon analogs and inhibitors of insulin secretion (eg, diazoxide, octreotide).

In accordance with one embodiment the pharmaceutical compositions disclosed herein are contemplated for use in methods of treating or preventing conditions of hypoglycemia, and more specifically treating or preventing atypical hypoglycemia, or medical conditions associated with hypoglycemia.

The compositions of the present disclosure can be administered using any standard routes of administration. Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. In one embodiment a pharmaceutical composition comprising a GLP-1 receptor antagonist of the present disclosure is formulated for subcutaneous administration or intravenous administration. In one embodiment the GLP-1 receptor antagonist of the disclosure, alone or in combination with other suitable components, can be prepared as an aerosol formulation to be administered via inhalation.

In one embodiment the composition is formulated for oral delivery by coformulation of a GLP-1 receptor antagonist of the present disclosure with an absorption enhancer, which can sufficiently augment the absorption of the peptide antagonist. Sodium N-[8-(2-hydroxybenzoyl)amino]caprylate (SNAC) is a delivery agent that has been reported to enhance the permeability of a diverse spectrum of molecules, including peptides, such as insulin, GLP-1, calcitonin, and other macromolecules such as heparin. In accordance with one embodiment pharmaceutical compositions are provided for oral delivery wherein the composition comprises a GLP-1 receptor antagonist of the present disclosure and SNAC, optionally wherein the pharmaceutical composition is formulated as a tablet.

### Acylation

In accordance with one embodiment, any of the peptides, dimers or multimers disclosed herein that exhibit GLP-1 antagonist activity can be further modified to have an improved therapeutic index and an in vivo extended time of action when administered to a warm blooded mammal including, for example, homo sapiens. More particularly, in one embodiment the peptides and dimers disclosed herein are modified by the covalent linkage of an alkyl or acyl group to the side chain of an amino acid, optionally a lysine, serine or cysteine, of the antagonist peptide, wherein the alkyl or acyl group is of sufficient size to bind to serum albumin with high affinity. In one embodiment the alkylated or acylated amino acid is located at the C-terminus of the GLP-1 antagonist peptide or dimer. In one embodiment the GLP-1 antagonist peptide comprises two acylated amino acids. In one embodiment one or more of the amino acids of the GLP-1 receptor antagonist peptide is acylated with a fatty acid or fatty diacid, optionally a C16-C18 fatty acid or C16-C18 fatty diacid. In accordance with one embodiment one or more lysine resides of the GLP-1 antagonist peptide or dimer disclosed herein is modified by the covalent linkage of a C16-C18 fatty acid or C16-C18 fatty diacid the side chain of a lysine, optionally via a spacer. In one embodiment the acylated lysine residue is the C-terminal amino acid of the GLP-1 antagonist peptide. In one embodiment an acylated amino acid is present at position 40 of the GLP-1 antagonist peptide and at a second position selected from position 7 or 12. In embodiments having two or more acylated amino acids, the acylated amino acids can be the same or different, and the linked acyl group can be the same or different, provided the acyl group is of sufficient size to bind serum albumin. In one embodiment the acylated amino acid is a lysine wherein the side chain is linked to a C16-C18 fatty acid or C16-C18 fatty diacid, optionally via a spacer.

In one embodiment, a C16-C18 fatty acid or C16-C18 fatty diacid is linked to the side chain of an amino acid via a spacer, wherein the spacer comprises a miniPEG, a gamma Glu, or any multimer or combination of miniPEG and/or gamma Glu. In one embodiment any of the GLP-1 receptor antagonist peptides disclosed herein may be modified to comprise an acylated amino acid, optionally at a position selected from 7, 12 and 40, in reference to the native sequence of exendin4. In one embodiment the acylated amino acid is a lysine residue having a C16 to C18 fatty acid or C16 to C18 fatty diacid linked to the lysine side chain via a spacer comprising the structure:
—[COCH₂(OCH₂CH₂)ₖNH]_{q}-(gamma glutamic acid)ₚ- wherein k is an integer selected from 2, 4, 6 or 8 and p and q are independently an integer selected from 1 or 2. In one embodiment the spacer is selected from the group consisting of
-(gamma glutamic acid)—[COCH₂(OCH₂CH₂)ₖNH]₂-(gamma glutamic acid)-, or
—[COCH₂(OCH₂CH₂)ₖNH]_{q}-(gamma glutamic acid)ₚ—[COCH₂(OCH₂CH₂)ₖNH]_{q}-(gamma glutamic acid)ₚ-, or
—[COCH₂(OCH₂CH₂)ₖNH]₂-(gamma glutamic acid)₂-, or
—[COCH₂(OCH₂CH₂)ₖNH]-(gamma glutamic acid)2—[COCH₂(OCH₂CH₂)ₖNH]-, or
-(gamma glutamic acid)₂—[COCH₂(OCH₂CH₂)ₖNH]₂-, or
-(gamma glutamic acid)—[COCH₂(OCH₂CH₂)ₖNH]-(gamma glutamic acid)ₚ—[COCH₂(OCH₂CH₂)ₖNH]-, or gamma glutamic acid, or a gamma glutamic acid-gamma glutamic acid dipeptide, or a gamma glutamic acid—[COCH₂(OCH₂CH₂)ₖNH]-gamma glutamic acid, wherein
k is an integer selected from the range of 1-8; and
q and p are independently an integer selected from the range of 1-8, optionally wherein k is 2 and q and p are independently 1 or 2. In one embodiment the spacer is
—[COCH₂(OCH₂CH₂)ₖNH]_{q}-(gamma glutamic acid)ₚ-, wherein
k is an integer selected from the range of 1-4; and
q and p are independently an integer selected from the range of 1-4, optionally wherein k is 2 and q and p are independently 1 or 2, optionally wherein k is 2 and q and p are both 1.

In one embodiment the GLP-1 receptor antagonist peptide comprises an acylated Lys, optionally located at the C-terminus of the peptide, wherein the side chain of the Lys is acylated with a C16-C18 diacid, via a spacer comprising the structure: —[COCH₂(OCH₂CH₂)ₖNH]_{q}-(gamma glutamic acid)ₚ-, wherein
k is an integer selected from the range of 1-4; and
q and p are independently an integer selected from the range of 1-4, optionally wherein k is 2 and q and p are independently 1 or 2, optionally wherein k is 2 and q and p are both 1. In one embodiment the side chain of the acylated Lys comprises the structure —[COCH₂(OCH₂CH₂)ₖNH]_{q}-(gamma glutamic acid)ₚ-COC₁₆H₃₂CO₂H, wherein k is 2 and p and q are independently 1 or 2. In one embodiment a GLP-1 receptor antagonist is provided comprising the sequence of DV(dW)RYLEEQAVREFIEWLVRGGPSSGAPPPSX₄₀R₂₀-, (SEQ ID NO: 27) or DVWRYLEEQAVREFIEWLVRGGPSSGAPPPSX₄₀R₂₀-, (SEQ ID NO: 28), wherein R₂₀ is COOH or CONH₂ and X₄₀ is a Lys acylated with —[COCH₂(OCH₂CH₂)ₖNH]_{q}-(gamma glutamic acid)ₚ-COC₁₄H₂₈CO₂H or —[COCH₂(OCH₂CH₂)ₖNH]_{q}-(gamma glutamic acid)ₚ-COC₁₆H₃₂CO₂H, wherein k is 2 and p and q are independently 1 or 2.

### Conjugates

In some embodiments any one of the GLP-1 antagonist peptides disclosed herein is conjugated to an immunoglobulin or portion thereof (e.g. variable region, CDR, or Fc region). Known types of immunoglobulins (Ig) include IgG, IgA, IgE, IgD or IgM. The Fc region is a C-terminal region of an Ig heavy chain, which is responsible for binding to Fc receptors that carry out activities such as recycling (which results in prolonged half-life), antibody dependent cell-mediated cytotoxicity (ADCC), and complement dependent cytotoxicity (CDC).

In some embodiments, any one of the GLP-1 antagonist peptides disclosed herein is conjugated to a hydrophilic moiety. Hydrophilic moieties can be covalently linked to the GLP-1 antagonist peptide under any suitable conditions used to react a protein with an activated polymer molecule. Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., alpha-iodo acetic acid, alpha-bromoacetic acid, alpha-chloroacetic acid). If attached to the peptide by reductive alkylation, the polymer selected should have a single reactive aldehyde so that the degree of polymerization is controlled. See, for example, Kinstler et al., Adv. Drug. Delivery Rev. 54:477-485 (2002); Roberts et al., Adv. Drug Delivery Rev. 54:459-476 (2002); and Zalipsky et al., Adv. Drug Delivery Rev. 16:157-182 (1995).

Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1,3-dioxolane, poly-1,3,6-trioxane, ethylene/maleic anhydride copolymer, poly (.beta.-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof. Dextrans are polysaccharide polymers of glucose subunits, predominantly linked by α1-6 linkages. Dextran is available in many molecular weight ranges, e.g., about 1 kD to about 100 kD, or from about 5, 10, 15 or 20 kD to about 20, 30, 40, 50, 60, 70, 80 or 90 kD. In one embodiment the hydrophilic moiety, e.g., polyethylene glycol chain, has a molecular weight selected from the range of about 500 to about 40,000 Daltons. In some embodiments the hydrophilic moiety is a polyethylene glycol chain having a molecular weight selected from the range of about 500 to about 5,000 Daltons, or about 1,000 to about 5,000 Daltons. In another embodiment the hydrophilic moiety, e.g., polyethylene glycol chain, has a molecular weight of about 10,000 to about 20,000 Daltons.

In one embodiment a conjugate derivative of the peptide of SEQ ID NO: 5 or SEQ ID NO: 23 is provided wherein a dipeptide is covalently linked via a peptide bond to the N-terminus of the peptide of SEQ ID NO: 5 or SEQ ID NO: 23, optionally wherein one of the amino acids of the dipeptide is an acylated amino acid. In one embodiment the dipeptide has the structure of X₇X₈-, wherein X₇ is an acylated amino acid and X₈ is any amino acid, optionally wherein X₇ is an acylated Lys or dLys and X₈ is Gly or a C₁-C₄ N-alkylated Gly, optionally wherein X₇ is a Lys or dLys acylated with a C14-C20 fatty acid or fatty diacid and X₈ is Gly or a C₁-C₄ N-alkylated Gly (optionally Gly or Sarcosine), optionally wherein X₇ is a Lys acylated with a C14-C20 fatty acid or fatty diacid via any of the spacers disclosed herein and X₈ is a C₁-C₄ N-alkylated Gly (optionally Gly or Sarcosine).

In accordance with one embodiment a conjugate derivative of any of the GLP-1 receptor antagonist peptides is provided wherein a self-cleaving dipeptide is covalently bound to an amino acid side chain amine or the N-terminal alpha amine of a GLP-1 antagonist peptide disclosed herein via an amide bond. In one embodiment the self-cleaving dipeptide is covalently bound to the N-terminal alpha amine of the GLP-1 antagonist peptide.

In exemplary aspects, the self-cleaving dipeptide comprises the structure: A-B wherein A is an amino acid or a hydroxy acid; and B is an N-alkylated amino acid linked to the GLP-1 antagonist peptide through an amide bond between A-B and an amine of the GLP-1 antagonist peptide, optionally wherein the chemical cleavage half-life (t1/2) of A-B from the GLP-1 antagonist peptide is at least about 1 hour to about 1 week in PBS under physiological conditions. As used herein the term “hydroxy acid” refers to an amino acid that has been modified to replace the alpha carbon amino group with a hydroxyl group.

In some embodiments the self-cleaving dipeptide has the general structure of:

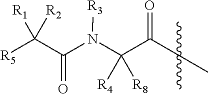

wherein
R₁-, R₂-, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃-, (C₁-C₄ alkyl)CONH₂-, (C₁-C₄ alkyl)COOH, (C₁-C₄ alkyl)NH₂-, (C₁-C₄ alkyl)NHC(NH₂⁺)NH₂-, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇-, (C₁-C₄ alkyl)(C₃-C₉ heteroaryl), and C₁-C₁₂ alkyl(W1)C₁-C₁₂ alkyl, wherein W1 is a heteroatom selected from the group consisting of N, S and O;
R₃ is selected from the group consisting of C₁-C₁₈ alkyl, (C₁-C₁₈ alkyl)OH, (C₁-C₁₈ alkyl)NH₂-, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇-, and (C₁-C₄ alkyl)(C₃-C₉ heteroaryl) or R₄ and R₃ together with the atoms to which they are attached form a pyrrolidine ring;
R₅ is NHR₆ or OH;
R₆ is H, C₁-C₈ alkyl; and
R₇ is selected from the group consisting of H and OH
wherein the chemical cleavage half-life (t_{1/2}) of A-B from said GLP-1 antagonist is at least about 1 hour to about 1 week in PBS under physiological conditions. In one embodiment the dipeptide A-B is covalently linked to the N-terminal alpha amine of the GLP-1 antagonist amino acid sequence.

In one embodiment the self-cleaving dipeptide has the general structure of:

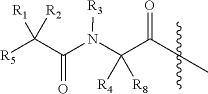

wherein
R₁ and R₈ are independently H or C₁-C₈ alkyl;
R₂ and R₄ are independently selected from the group consisting of H, C₁-C₈ alkyl, (C₁-C₄ alkyl)OH, (C₁-C₄ alkyl)SH, (C₂-C₃ alkyl)SCH_{3,} (C₁-C₄ alkyl)CONH₂-, (C₁-C₄ alkyl)COOH, (C₁-C₄ alkyl)NH₂-, and (C₁-C₄ alkyl)(C₆ aryl)R₇-;
R₃ is C₁-C₆ alkyl;
R₅ is NH₂-; and
R₇ is selected from the group consisting of hydrogen, and OH.

In accordance with one embodiment any of the GLP-1 antagonist peptides and dimers disclosed herein can be further modified by linkage to a self-cleaving dipeptide wherein an amino acid of the dipeptide is acylated with a fatty-acyl group of sufficient size to bind serum albumin with high affinity.

In one embodiment amino acid “A” of the self-cleaving dipeptide “A-B” is a lysine residue acylated with a C16-C30 fatty acid or C16-C30 diacid. In one embodiment A and B are selected to provide a chemical cleavage half-life (t1/2) of A-B from the GLP-1 antagonist peptides or dimers disclosed herein of at least about 24 hours to about 240 hours, about 48 hours to about 168 hours, or about 48 to about 120 hours, or about 70 to about 100 hours in standard PBS solution under physiological conditions.

In one embodiment the self-cleaving dipeptide has the general structure of :

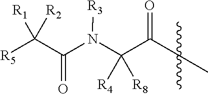

wherein
R₁, comprises a side chain selected from the group consisting of C₁-C₈ alkyl, (C₁-C₄ alkyl)₀H, alkyl)SH, (C₁-C₄ alkyl)COOH, and (C₁-C₄ alkyl)NH₂-, optionally wherein a C16-C18 fatty acid or C16-C18 diacid is covalently linked to said side chain, optionally via any of the spacers disclosed herein, including spacers selected from the group consisting of a gamma glutamic acid, a gamma glutamic acid-gamma glutamic acid dipeptide, —[COCH₂(OCH₂CH₂)ₖNH]_{q}-(gamma glutamic acid)ₚ, and a gamma glutamic acid—[COCH₂(OCH₂CH₂)ₖNH]_{q}-gamma glutamic acid, wherein
k is an integer selected from the range of 1-8; and
q and p are independently an integer selected from the range of 1-8, optionally wherein k is 2 and q and p are independently 1 or 2;
R₂-, R₄ and R₈ are independently H, or C₁-C₄ alkyl;
R₃ is C₁-C₆ alkyl; and
R₅ is NH₂-.

In one embodiment the self-cleaving dipeptide has the general structure of:

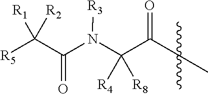

wherein
R₁ is H, C₁-C₄ alkyl, (C₁-C₄ alkyl)OH or (C₁-C₄ alkyl)NH₂-;
R₂ is H,
R₃ is C₁-C₄ alkyl;
R₄ is H or C₁-C₄ alkyl;
R₅ is NH₂; and
R₈ is hydrogen.

In accordance with one embodiment the self-cleaving dipeptide A-B comprises an acylated amino acid residue as “A” and an N-alkylated Gly residue as “B”, wherein the “B” amino acid is linked to the N-terminal alpha amine of the GLP-1 receptor antagonist peptide via an amide bond, optionally wherein said Lys residue is in the D-conformation. In one embodiment the acylated amino acid “A” of the A-B dipeptide is an amino acid having the general structure of

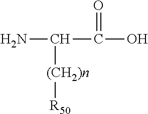

wherein n is an integer selected from the range of 1-4 and R50 is selected from the group consisting of NH-CO(CH₂)₁₄₋₂₀COOH, NH-[spacer]-CO(CH₂)₁₄₋₂₀COOH, S(CH₂)₁₄₋₂₀COOH and S-[spacer]-CO(CH₂)₁₄₋₂₀COOH, wherein the [spacer] is any of the spacers disclosed herein. In one embodiment the acylated amino acid of A is independently selected from lysine, d-lysine, ornithine, cysteine or homocysteine wherein the side chain of said acylated amino acid is covalently linked to a C16-C22 fatty acid or C16-C22 diacid optionally through a spacer comprising an amino acid or dipeptide. In one embodiment the spacer comprises a gamma glutamic acid. In one embodiment the spacer comprises multiple units of gamma glutamic acid and miniPeg polymers in any combination. In one embodiment the optional spacer comprises two gamma glutamic acids, optionally wherein the two gamma glutamic acids are joined to one another via an intervening functionalized miniPEG polymer, [COCH₂(OCH₂CH₂)ₖHN]q, wherein k and q are each integers independently selected from 1, 2, 3, 4, 5, 6, 7 or 8.

In one embodiment the self-cleaving dipeptide has the general structure of :

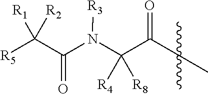

wherein
R₁ is (C₄ alkyl)NH₂ or (C₄ alkyl)NH(mPeg-γE-diacid)-C18;
R₂-, R₄ and R₈ are each H;
R₃ is C₁-C₄ alkyl; and
R₅ is NH₂-, optionally wherein the first amino acid of the self-cleaving dipeptide is in the D-conformation.

In one embodiment the GLP-1 antagonist peptides and dimers disclosed herein are covalently linked to a self-cleaving dipeptide of the structure:

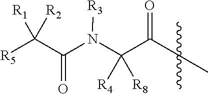

wherein
R₁-, is a side chain selected from the group consisting of C₁-C₁₈ alkyl, (C₁-C₄ alkyl)OH, (C₁-C₄ alkyl)SH, (C₁-C₄ alkyl)COOH, and (C₁-C₄ alkyl)NH₂-, optionally wherein a C16-C20 fatty acid or a C16-C20 diacid is covalently linked to said side chain;
R_{2,} R₄ and R₈ are independently H, or C₁-C₄ alkyl;
R₃ is C₁-C₄ alkyl, or R₄ and R₃ together with the atoms to which they are attached form a pyrrolidine ring; and
R₈ is NH₂-, with the proviso that when R₄ and R₃ together with the atoms to which they are attached form a pyrrolidine ring, R₂ is not H.

In a further embodiment the self-cleaving dipeptide has the structure of

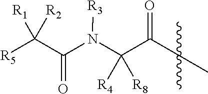

wherein R₁-, is (C₁-C₄ alkyl)NH-CO(CH₂)₁₄-₂₀CH₃-, (C₁-C₄ alkyl)NH-[spacer]-CO(CH₂)₁₄-₂₀CH₃-, (C₁-C₄ alkyl)NH-CO(CH₂)₁₄-₂₀COOH or (C₁-C₄ alkyl)NH-[spacer]-CO(CH₂)₁₄-₂₀COOH; R₂ and R₈ are each H; R₄ is H or CH₃-; R₃ is C₁-C₄ alkyl and R₅ is NH₂-, optionally wherein the first amino acid of the self-cleaving dipeptide is an amino acid in the D-stereochemical configuration and the spacer is selected from any of the spacers disclosed herein. In one embodiment R₂-, R₄ and R₈ are each H. In one embodiment R₁-, is (C₁-C₄ alkyl)NH-CO(CH₂)₁₆COOH or (C₁-C₄ alkyl)NH-[spacer]-CO(CH₂)₁₆COOH, R₂-, R₄ and R₈ are each H and R₃ is C₁-C₄ alkyl, optionally CH₃-, optionally wherein the spacer comprises the structure:
—[COCH₂(OCH₂CH₂)ₖNH]_{q}-(gamma glutamic acid)ₚ-
wherein k is 2, and p and q are independently an integer selected from 1 or 2.

In accordance with one embodiment the self-cleaving dipeptide comprises an acylated amino acid as the first amino acid, wherein the side chain of the acylated amino acid is acylated with a C16-C20 fatty acid or C16-C20 diacid, optionally wherein the acylated amino acid is selected from C16-C20 acylated lysine, C16-C20 acylated ornithine, C16-C20 acylated cysteine and C16-C20 acylated homocysteine, optionally wherein the acylated amino acid of the dipeptide is a C16-C20 acylated Lys, optionally wherein the first amino acid of the self-cleaving dipeptide is an amino acid in the D-stereochemical configuration.

### EMBODIMENTS

In accordance with embodiment 1, a GLP-1 receptor antagonist is provided, wherein said antagonist comprises the amino acid sequence of R₁₀-DVX₁₁X₁₂YLX₁₅X₁₆QAX₁₉X₂₀EFX₂₃EWLVRGGPSSGAPPPSX₄₀-R₂₀ SEQ ID NO: ₂₃), wherein
R₁₀ is NH₂ or an N-terminal extension of 1 or 2 amino acids, wherein one of the amino acids of the N-terminal extension is acylated with a C14-C20 fatty acid or diacid, optionally via a spacer, optionally wherein R₁₀ is a dipeptide of the structure: X₇X₈-, wherein X₇ is an acylated amino acid, optionally acylated Lys or acylated dLys, and X₈ is Gly or a C₁-C₄ N-alkylated Gly;
X₁₁ is Trp, dTrp or Ser;
X₁₂ is Arg or an acylated amino acid, optionally acylated Lys;
X₁₅ is Glu or dGlu;
X₁₆ is Glu, dGlu, Asp, homoglutamic acid or homocysteic acid;
X₁₉ is Val, cyclopropane, cyclopentane, cyclohexane or phenyl glycine;
X₂₀ is Arg, homolysine or citrulline;
X₂₃ is Ile, or dIle; and
X₄₀ is an acylated amino acid, optionally an acylated Lys; and
R₂₀ is COOH or CONH₂-, optionally wherein 1, 2 or 3 amino acids selected from positions 7, 10, 13, or 16 are substituted with Trp or dTrp, with the proviso that when one of X₁₀ or X₁₁ is Trp or dTrp then the other is not Trp or dTrp and that R₁₀ and X₁₂ cannot both comprise an acylated amino acid; optionally wherein 1, 2 or 3 amino acids at any of positions 16, 18, 19, 24, 26, or 28 are substituted with Aib.

In accordance with embodiment 2 a GLP-1 antagonist of embodiment 1 is provided wherein said antagonist comprises the amino acid sequence of R₁₀-DVX₁₁X₁₂YLX₁₅X₁₆QAX₁₉X₂₀EFX₂₃EWLVRGGPSSGAPPPSX₄₀-R₂₀ SEQ ID NO: 23), wherein
R₁₀ is NH₂ or X₇X₈-, wherein X₇ is an amino acid acylated with a C14-C20 fatty acid or diacid, optionally via a spacer and X₈ is Gly or a C₁-C₄ N-alkylated Gly;
X₁₁ is Trp, dTrp or Ser;
X₁₂ is Arg or an acylated amino acid, optionally acylated Lys;
X₁₅ is Glu or dGlu;
X₁₆ is Glu, dGlu, Asp, homoglutamic acid or homocysteic acid;
X₁₉ is Val, cyclopropane, cyclopentane, cyclohexane or phenyl glycine;
X₂₀ is Arg, homolysine or citrulline;
X₂₃ is Ile, or dIle; and
X₄₀ is an acylated amino acid, optionally an acylated Lys; and
R₂₀ is COOH or CONH₂-, optionally wherein 1, 2 or 3 amino acids at any of positions 16, 18, 19, 24, 26, or 28 are substituted with Aib.

In accordance with embodiment 3 a GLP-1 antagonist of embodiment 1 or 2 is provided wherein each acylated amino acid of the GLP-1 antagonist is an acylated Lys.

In accordance with embodiment 4 a GLP-1 antagonist of any one of embodiments 1-3 is provided wherein X₇ is an acylated Lys and X₁₂ is Arg.

In accordance with embodiment 5 a GLP-1 antagonist of any one of embodiments 1-3 is provided wherein R₁₀ is NH₂ and X₁₂ is an acylated Lys.

In accordance with embodiment 6 a GLP-1 antagonist of any one of embodiments 1-3 is provided wherein X₇ is NH₂ and X₁₂ is Arg.

In accordance with embodiment 7 a GLP-1 antagonist of embodiment 1 is provided the antagonist comprises an amino acid sequence selected from any one of SEQ ID NO: 5 though SEQ ID NO: 96, or any combination thereof.

In accordance with embodiment 8 a GLP-1 antagonist of any one of embodiments 1-7 is provided wherein the acylated amino acids of said GLP-1 antagonist are independently a Lys residue acylated with a C16-C18 fatty acid or C16-C18 fatty diacid directly linked to the Lys side chain or optionally via a spacer comprising a
i) gamma glutamic acid,
ii) minipeg polymer: —[COCH₂(OCH₂CH₂)ₖNH]-, wherein k is 2, 4, 6 or 8,
iii) or any multiplicity or combination of i) and/or ii).

In accordance with embodiment 9 a GLP-1 antagonist of any one of embodiments 1-8 is provided wherein said antagonist comprises the amino acid sequence of
DX₁₀X₁₁RYLX₁₅X₁₆QAVREFX₂₃EWLVRGGPSSGAPPPSX₄₀R₂₀ (SEQ ID NO: 5), wherein
X₁₀ is Trp, dTrp or Val;
X₁₁ is Trp, dTrp or Ser;
X₁₅ is Glu or dGlu
X₁₆ is Trp, dTrp, dGlu or Glu;
X₂₃ is Ile or dIle;
X₄₀ is an acylated amino acid; and
R₂₀ is COOH or CONH₂-, optionally wherein the peptide comprises one or more substitutions of Aib at any of positions 16, 18, 19, 24, 26 or 28, or optionally a substitution of an acylated Lys at position 12, wherein said position number is relative to the native Exendin4 amino acid sequence.

In accordance with embodiment 10 a GLP-1 antagonist of any one of embodiments 1-9 is provided wherein X₁₁ is Trp or dTrp.

In accordance with embodiment 11 a GLP-1 antagonist of any one of embodiments 1-10 is provided wherein an amino acid at position at any of positions 16, 18, 19, 24, 26 or 28 of SEQ ID NO: 5 is substituted with Aib, optionally wherein an Aib is substituted at position 18.

In accordance with embodiment 12 a GLP-1 antagonist of any one of embodiments 1-11 is provided wherein the amino acid at position 12 is substituted with an acylated Lys.

In accordance with embodiment 13 a GLP-1 antagonist of any one of embodiments 1-11 is provided wherein
X₁₅ is dGlu;
X₁₆ is Glu; and
X₂₃ is Ile.

In accordance with embodiment 14 a GLP-1 antagonist of any one of embodiments 1-11 is provided wherein
X₁₅ is Glu;
X₁₆ is Glu; and
X₂₃ is Ile.

In accordance with embodiment 15 a GLP-1 antagonist of any one of embodiments 1-14 is provided wherein X₄₀ is an amino acid having an acyl group linked to the side chain of the amino acid, optionally via a spacer.

In accordance with embodiment 16 a GLP-1 antagonist of any one of embodiments 1-15 is provided wherein X₄₀ is an amino acid comprising a structure of Formula I (optionally, Lys), Formula II (optionally, Cys), or Formula III (optionally, Ser), wherein each of Formulae I, II, and III, is:

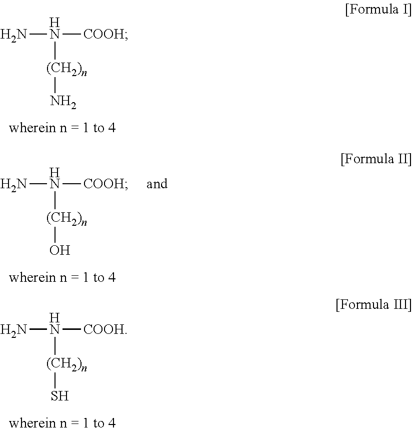

In accordance with embodiment 17 a GLP-1 antagonist of any one of embodiments 1-16 is provided wherein X₄₀ is an acylated lysine.

In accordance with embodiment 18 a GLP-1 antagonist of any one of embodiments 1-17 is provided wherein the acyl group of the acylated amino acid is selected from (C₁-C₄ alkyl)NH-CO(CH₂)₁₄₋₂₀CH₃-, (C₁-C₄ alkyl)NH-[spacer]-CO(CH₂)₁₄₋₂₀CH₃-, (C₁-C₄ alkyl)NH-CO(CH₂)₁₄₋₂₀COOH or (C₁-C₄ alkyl)NH-[spacer]-CO(CH₂)₁₄₋₂₀COOH.

In accordance with embodiment 19 a GLP-1 antagonist of any one of embodiments 1-18 is provided wherein the acyl group of the acylated amino acid is covalently linked to the amino acid side chain of the acylated amino acid via a spacer.

In accordance with embodiment 20 a GLP-1 antagonist of any one of embodiments 1-19 is provided wherein the spacer is an amino acid or dipeptide.

In accordance with embodiment 21 a GLP-1 antagonist of any one of embodiments 1-20 is provided wherein the spacer comprises a
i) gamma glutamic acid,
ii) minipeg polymer: —[COCH₂(OCH₂CH₂)ₖNH]-, wherein k is 2, 4, 6 or 8,
iii) or any multiplicity or combination of i) and/or ii).

In accordance with embodiment 22 a GLP-1 antagonist of any one of embodiments 1-11 is provided wherein the acylated amino acid is linked to a C16 to C18 fatty acid or C16 to C18 fatty diacid, optionally wherein the acid or diacid is linked via a spacer comprising the structure:
-(gamma glutamic acid)ₚ—[COCH₂(OCH₂CH₂)ₖNH]_{q}-(gamma glutamic acid)ₙ- or
—[COCH₂(OCH₂CH₂)ₖNH]_{q}-(gamma glutamic acid)ₚ—[COCH₂(OCH₂CH₂)ₖNH]ₘ-;
wherein k is an integer selected from 2, 4 or 8, m and n are independently an integer selected from 0, 1 or 2, and p and q are independently an integer selected from 1, 2, 4 or 8.

In accordance with embodiment 23 a GLP-1 antagonist of any one of embodiments 1-22 is provided wherein the acylated amino acid is Lys having a C16 to C18 fatty acid linked to the lysine side chain via a spacer comprising the structure:
—[COCH₂(OCH₂CH₂)ₖNH]_{q}-(gamma glutamic acid)ₚ-
wherein k is 2, and p and q are independently an integer selected from 1 or 2.

In accordance with embodiment 24 a GLP-1 antagonist of any one of embodiments 1-23 is provided wherein
X₁₁ is Trp or dTrp;
X₁₅ is Glu or dGlu;
X₁₆ is Glu or dGlu;
X₂₃ is Ile or dIle; and
X₄₀ is Lys acylated with a C16 or C18 diacid, optionally wherein the diacid is linked via a spacer comprising the structure:
—[COCH₂(OCH₂CH₂)ₖNH]_{q}-(gamma glutamic acid)ₚ-
wherein k is 2, and p and q are independently an integer selected from 1 or 2.

In accordance with embodiment 25 a GLP-1 antagonist of any one of embodiments 1-24 is provided wherein R₂₀ is CONH₂-.

In accordance with embodiment 26 a derivative of the GLP-1 antagonist of any one of the claims 9-25 is provided further comprising a dipeptide A-B:

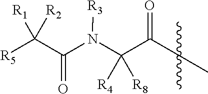

linked to said GLP-1 antagonist through an amide bond wherein
R₁-, R₂-, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃-, (C₁-C₄ alkyl)CONH₂-, (C₁-C₄ alkyl)COOH, (C₁-C₄ alkyl)NH₂-, (C₁-C₄ alkyl)NHC(NH₂⁺)NH₂-, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇-, (C₁-C₄ alkyl)(C₃-C₉ heteroaryl), and C₁-C₁₂ alkyl(W1)C₁-C₁₂ alkyl, wherein W1 is a heteroatom selected from the group consisting of N, S and O;
R₃ is selected from the group consisting of C₁-C₁₈ alkyl, (C₁-C₁₈ alkyl)OH, (C₁-C₁₈ alkyl)NH₂-, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇-, and (C₁-C₄ alkyl)(C₃-C₉ heteroaryl) or R₄ and R₃ together with the atoms to which they are attached form a pyrrolidine ring;
R₅ is NHR₆ or OH;
R₆ is H, C₁-C₈ alkyl; and
R₇ is selected from the group consisting of H and OH
wherein the chemical cleavage half-life (t_{1/2}) of A-B from said GLP-1 antagonist is at least about 1 hour to about 1 week in PBS under physiological conditions.

In accordance with embodiment 27 a GLP-1 antagonist of embodiments 26 is provided wherein the dipeptide A-B is covalently linked to the N-terminal alpha amine of the GLP-1 antagonist amino acid sequence.

In accordance with embodiment 28 a GLP-1 antagonist of any one of embodiments 26-27 is provided wherein
R₁ and R₈ are independently H or C₁-C₈ alkyl;
R₂ and R₄ are independently selected from the group consisting of H, C₁-C₈ alkyl, (C₁-C₄ alkyl)OH, (C₁-C₄ alkyl)SH, (C₂-C₃ alkyl)SCH₃-, (C₁-C₄ alkyl)CONH₂-, (C₁-C₄ alkyl)COOH, (C₁-C₄ alkyl)NH₂-, and (C₁-C₄ alkyl)(C₆ aryl)R₇-;
R₃ is C₁-C₆ alkyl;
R₅ is NH₂-; and
R₇ is selected from the group consisting of hydrogen, and OH.

In accordance with embodiment 29 a GLP-1 antagonist of any one of embodiments 26-27 is provided wherein
R₁-, comprises a side chain selected from the group consisting of C₁-C₈ alkyl, (C₁-C₄ alkyl)OH, (C₁-C₄ alkyl)SH, (C₁-C₄ alkyl)COOH, and (C₁-C₄ alkyl)NH₂-, optionally wherein a C16-C30 fatty acid or C16-C30 diacid is covalently linked to said side chain, optionally via a spacer selected from the group consisting of a gamma glutamic acid, a gamma glutamic acid-gamma glutamic acid dipeptide, and a gamma glutamic acid-[COCH₂(OCH₂CH₂)ₖNH]_{q}-gamma glutamic acid, wherein
k is an integer selected from the range of 1-8; and
q is an integer selected from the range of 1-8, optionally wherein k is 2 and q is selected from the range of 1-8;
R₂-, R₄ and R₈ are independently H, or C₁-C₄ alkyl;
R₃ is C₁-C₆ alkyl; and
R₅ is NH₂-.

In accordance with embodiment 30 a GLP-1 antagonist of any one of embodiments 26-29 is provided wherein
R₁ is H, C₁-C₄ alkyl, (C₁-C₄ alkyl)OH or (C₁-C₄ alkyl)NH₂-;
R₂ is H,
R₃ is C₁-C₄ alkyl;
R₄ is H, or C₁-C₄ alkyl;
R₅ is NH₂-; and
R₈ is hydrogen.

In accordance with embodiment 31 a GLP-1 antagonist of any one of embodiments 26-30 is provided wherein said dipeptide A-B comprises an acylated Lys residue and an N-alkylated Gly residue, wherein said Lys and N-alkylated Gly residues are linked via a peptide bond, optionally wherein said Lys residue is in the D-conformation.

In accordance with embodiment 32 a GLP-1 antagonist of any one of embodiments 26-30 is provided wherein
R₁ is (C₄ alkyl)NH₂ or (C₄ alkyl)NH(mPeg-γE-diacid)-C18;
R₂-, R₄ and R₅ are each H;
R₃ is C₁-C₄ alkyl; and
R₅ is NH₂-;
R₅ is an amine.

In accordance with embodiment 33 a pharmaceutical composition comprising a GLP-1 antagonist or derivatives of any one of claims 1-32 and a pharmaceutically acceptable carrier, diluent, or excipient is provided.

In accordance with embodiment 34 a method of treating a patient suffering from atypical hypoglycemia is provided wherein said method comprises the step of administering to a patient in need thereof a pharmaceutical composition of embodiment 33 in an amount effective to elevate blood glucose levels.

### Example 1

Ex-4 (9-39)a (SEQ ID NO: 2) is an established antagonist of the GLP-1 receptor. However, its use as a therapeutic agent in humans is limited due to its nonhuman origin and its relatively short in vivo duration of action. Comparable N-terminal shortening of human GLP-1 lessens agonism but does not provide a high potency antagonist. Through a series of GLP-1/Ex-4 hybrid peptides, the minimal structural changes required to generate a pure GLP-1-based antagonist were identified as Glu16, Val19, and Arg20, yielding an antagonist of approximately 3-fold greater in vitro potency compared with Ex-4 (9-39)a. Site-specific acylation of the human-based antagonist yielded a peptide “9-40 Jant4 K₄₀[C16] (SEQ ID NO: 3) of increased potency as a GLP-1 receptor antagonist and 10-fold greater selectivity relative to the GIP receptor (Patterson, et al, ACS Chem. Biol. 2011, 6, 135-145).

As disclosed herein, variants of 9-40 Jant4-K₄₀ have been prepared to provide a further improved GLP-1 receptor antagonist. Peptide 9-40 Jant4-K₄₀ is acylated at position 40 by direct linkage of a C16 acyl group to the Lys side chain. Variants have been prepared inserting a spacer between the Lys side chain and the C16 acyl group. Table 2 presents the GLP-1 antagonist activity and solubility of these acylation variants.

Various amino acid substitutions were made to the primary sequence of 9-40Jant4-K₄₀ (DVSSYLEEQAVREFIAWLVKGGPSSGAPPPSK;SEQ ID NO: 3) and the GLP-1 antagonist activity and solubility of these variants is provided in Table 3.

Table 4 provides data regarding the effect of Aib substitutions on the activity and solubility of Jant4-K₄₀ (SEQ ID NO: 3) variants. Table 5 provides data regarding the effect of d-AA substitutions on the solubility of Jant4-K₄₀ (C16) (SEQ ID NO: 3) variants and Table 6 presents data regarding Trp substitution at position 3 of Jant4-K₄₀ (SEQ ID NO: 3).

### Materials and Methods

### Fmoc Synthesis

Peptides were prepared by automated Fmoc/t-Bu solid-phase methodology employing a Symphony peptide synthesizer (Peptide Tech-nology, Tucson, AZ) starting with Wang resin (AAPPtec, Louisville, KY) and 6-Cl-HOBt/DIC activation. All conventional residues were purchased from Midwest Biotech (Fisher, IN), 6-Cl-HOBt and DIC was obtained from AAPPtec (Louisville, KY). Peptides were cleaved from the resin and de-protected by treatment with TFA containing 2.5% TIS, 2.5% H2O, 1.5% methanol, 2.5% phenol, 0.5% DODT and 0.5% of dimethylsulfoxide. Peptide was precipitated with cold ethyl ether from a filtered TFA solution according to standard procedure. The fatty-acylation of peptides was performed on resin with tenfold excess of Fmoc-Glu-OtBu/DEPBT/DIEA, repeated for double coupling, followed by tenfold excess of palmitic acid or another fatty acid/DEPBT/DIEA.

### Antagonist Acylation

Palmitic acid was introduced to synthesized antagonist peptides using an orthoginal solid-phase protection scheme. Boc synthesis was utilized for peptide synthesis, allowing selective introduction of base-sensitive side-chain protected Lys(Fmoc)-OH at Lys40. The fully protected peptides were treated on resin with 20% piperdine inDMF (v/v) for 30 min to remove the Lys40 side-chain Fmoc group. Amide bond formation was facilitated with excess fatty acid and 5 equiv of benzotriazol-1-yl-oxytripyrrolidinophosphonium hexafluorophosphate (PyBOP) (Fluka) in DMF/DIEA (4:1 v/v) for approximately 18 h. Ninhydrin testing was used to monitor reaction progress, and acylation was confirmed after peptide cleavage by ESI mass spectrometry.

### Peptide Purification

Reversed-phase HPLC (RP-HPLC) was used for peptide purification. A C18 stationary phase (Vydac 218TP, 250 mm_22 mm, 10 μm) was employed with a linear acetonitrile gradient in 0.1% trifluoroacetic acid during the preparative RP-HPLC purification. Analytical analysis was performed on peak fractions by employing RP-HPLC with a C8 column (Zorbax 300SB, 4.6 min×50 mm, 3.5 μm). Peptide identity and purity was assessed by analytical RP-HPLC and ESI- or MALDI-mass spectrometry. All peptides were found to have the correct molecular weight and were approximately 95% pure. Lyophilized peptides were stored at 4° C.

GLP-1 Receptor-Mediated cAMP Induction

The ability of peptides to stimulate or block cAMP induction at the GLP-1 receptor was examined by a luciferase-based reporter gene assay. Cotransfection of HEK 293 cells with the human GLP-1 receptor (Open Biosystems) and a cAMPinducible (cAMPresponsive element) luciferase gene constituted the cellular construct where receptor activation could be measured. Bioassays were performed by first serum depriving the cells for 16 h in 0.25% bovine growth serum (HyClone)-supplemented Dulbecco's modified Eagle's medium (Invitrogen) and then adding serial dilutions of the peptides over the appropriate concentration ranges in 96-well poly-D-lysine-coated plates (BD Biosciences). For antagonism assays, a constant concentration of GLP-1 (0.05 nM) was added to the assay plate after the diluted peptides. Incubation continued for 5 h at 37° C., 5% CO₂ and was followed by the addition of an equivalent volume (100 μL) of LucLite luminescence substrate reagent (Perkin-Elmer). MicroBeta 1450 liquid scintillation counting (Perkin-Elmer) quantified the luminescence signal in counts per second (cps) after shaking the plate at 600 rpm for 3 min. Data was plotted using Origin software (OriginLab) and the effective concentration 50 (EC₅₀) or inhibitory concentration 50 (IC₅₀) was determined by sigmoidal fitting. Potency was determined by comparative analysis of relative EC₅₀ or IC₅₀ values. Each experiment was repeated at least three times with each sample assayed in duplicate.

### Animals

C57B1/6 mice were obtained from Jackson Laboratories. Mice were single- or group-housed on a 12:12 h light-dark cycle at 22° C. with free access to food and water. All studies were approved by and performed according to the guidelines of the Institutional Animal Care and Use Committee of the University of Cincinnati.

### Glucose Tolerance Test (GTT)

For the determination of glucose tolerance, mice were subjected to 6 h of fasting and injected intraperitoneally with glucose. Injections consisted of 1.5 g glucose per kg body weight (25% w/v D-glucose (Sigma) in 0.9% w/v saline). Tail blood glucose levels (mg dL-1) were measured using a hand-held glucometer (FreeStyle Freedom Lite) before 0 min and at 15, 30, 60, and 120 min after injection.

### Acute In Vivo Study

Mice received a subcutaneous injection of peptide (in PBS) either 1, 4, 8, 24, 48, 72, or 120 at varying doses before being challenged with an IP injection of dipeptidyl peptidase-IV-protected Ex-4 (0.65 nmol kg−1). A GTT was performed fifteen minutes after the GLP-1 injection. Tail blood glucose values were obtained as described above.

TABLE 2
C16 Linker optimization/analysis on Jant 4-K₄₀ sequence (SEQ ID NO: 3)

| | | | | | Solubility |
|---|---|---|---|---|---|
| | | | | | [mg/ml] |
| | | | | | 80 mM |
| | 9-40amide; Jant 4 | Bioactivity | | | AmBicarb |
| # | K40[X] | IC₅₀ | SDV | N | pH 7.7 |
| 95 | Free Amine | 281 | 96 | 3 | 12.7 |
| 47 | C16 | 14 | 7 | 15 | 6.1 |
| 52 | γE-C16 | 12 | 3 | 7 | >20 |
| 53 | γE₂-C16 | 13 | 5 | 10 | >20 |
| 54 | γE₄-C16 | 21 | 8 | 8 | >20 |
| 55 | γE₆-C16 | 22 | 15 | 5 | >20 |
| 48 | (miniPEG)₂-γE-C16 | 9 | 4 | 7 | 9.0 |
| 56 | miniPEG-γE-C16 | 14 | 2 | 3 | >20 |
| 57 | gE-(miniPEG)₂-gE- | 9 | 6 | 5 | >25 |
| | C16 | | | | |
| 58 | (miniPEG)₂-gE₂-16 | 11 | 6 | 5 | >25 |
| 59 | miniPEG-gE- | 11 | 7 | 5 | >25 |
| | miniPEG- gE-C16 | | | | |
| 60 | miniPEG-gE₂- | 5 | 2 | 5 | >25 |
| | miniPEG-C16 | | | | |
| 61 | gE₂-(miniPEG)₂-C16 | 6 | 2 | 4 | >25 |
| 62 | gE-miniPEG-gE- | 7 | 3 | 4 | >25 |
| | miniPEG-C16 | | | | |
| 63 | DiAcidC18 | 114 | 65 | 16 | >25 |
| 64 | gE₂-DiAcidC18 | 841 | 361 | 7 | >25 |
| 65 | gE₄₋DiAcidC18 | 1071 | 624 | 8 | >25 |
| 66 | gE₆-DiAcidC18 | 1464 | 751 | 6 | >25 |
| 67 | (miniPEG)₂-gE- | 579 | 376 | 12 | >25 |
| | DiAcidC18 | | | | |
| 68 | miniPEG-gE- | 718 | 130 | 3 | >25 |
| | DiAcidC18 | | | | |

TABLE 3
Amino acid substitution variants of Jant4-K₄₀ (SEQ ID NO: 3)

| | | | | | | Solubility |
|---|---|---|---|---|---|---|
| | | | | | | 80 mM |
| | | | Bioactivity | | | AmBicarb |
| # | 9-40; Jant4 | K40[C16] | IC₅₀ | SDV | N | pH 7.7 |
| 47 | | -amide | 14 | 7 | 15 | 6.1 |
| 69 | R28 | -amide | 16 | 1 | 3 | 13.7^{‡} |
| 70 | E24, R28 | -amide | 14 | 4 | 4 | >20 |
| 71 | E17, R28 | -amide | 18 | 7 | 4 | >20 |
| 72 | E17, E24, R28 | -amide | 19 | 6 | 4 | >20 |
| 73 | R12, R28 | -amide | 27 | 17 | 4 | <0.1 |
| 74 | R12, E24, R28 | -amide | 15 | 3 | 4 | >20 |
| 75 | R12, E17, E24, R28 | -amide | 19 | 5 | 4 | 18.5 |
| 76 | Exendin K40 | -amide | 6 | 1 | 3 | 18.6 |
| 77 | R28 ^{¥} | -ACID | 13 | 2 | 5 | >20 |
| 78 | E24, R28 ^{¥} | -ACID | 19 | 13 | 13 | >20 |
| 16 | R12, E24, R28 ^{¥} | -ACID | 18 | 12 | 13 | >20 |
| 79 | E24, R27, V28 ^{¥} | -ACID | 115 | 37 | 10 | >20 |
| 80 | E24, R27, Aib28 ^{¥} | -ACID | 106 | 14 | 4 | >20 |

^{¥} K40[miniPEG-gE-C16]
^{‡}Highly viscous solution

TABLE 4
Effect of Aib on activity & solubility of Jant4-K₄₀ (SEQ ID NO: 3)

| | 9-40am; | | | | Solubility | Solubility |
|---|---|---|---|---|---|---|
| | Jant4 | Bioactivity | | | [mg/mL] 80 mM | [mg/mL] |
| # | K40[C16] | IC₅₀ | SDV | N | AmBicarb pH 7.7 | PBS pH 7.4 |
| 81 | Aib27 | 23 | 10 | 4 | 19.0 (6.1) | 1.6 |
| 43 | K27, Aib28 | 13 | 13 | 10 | 18.9 (5.1) | 0.2 |
| 82 | K26, Aib28 | 6 | 2 | 4 | 14.2 (6.0) | 0.1 |
| 44 | Aib26 | 8 | 1 | 4 | 19.0 (5.7) | 0.1 |
| 83 | Aib24 | 14 | 6 | 4 | 12.6 (4.2) | 0.3 |
| 45 | Aib19 | 8 | 2 | 4 | 11.1 (1.6) | 0.4 |
| 46 | Aib18 | 7 | 2 | 3 | 17.5 (3.5) | 0.7 |
| 84 | Aib16 | 30 | 13 | 3 | 5.2 (0.2) | 0.2 |

TABLE 5
Improving solubility of Jant4-K₄₀ (C16) (SEQ ID NO: 3) with d-AA

| | | | | | Solubility [mg/mL] |
|---|---|---|---|---|---|
| | 9-40am; Jant4 | Bioactivity | | | 80 mM AmBicarb |
| # | K40[C16) | IC₅₀ | SDV | N | pH 7.7 |
| 49 | d-Glu¹⁵ | 23 | 12 | 7 | >20 |
| 85 | d-Glu¹⁶ | 41 | 20 | 7 | 10.7 |
| 86 | d-Gln¹⁷ | 41 | 24 | 6 | 2.2 |
| 50 | d-Val¹⁹ | 124 | 86 | 6 | >20 |
| 87 | d-Arg²⁰ | 16 | 6 | 4 | 0.8 |
| 88 | d-Glu²¹ | 27 | 13 | 4 | 1.7 |
| 51 | d-Ile²³ | 31 | 12 | 5 | >20 |

TABLE 6
Trp substitution at position 3 of Jant4-K₄₀ (SEQ ID NO: 3)

| SEQ ID | | |
|---|---|---|
| NO: # | Structure | IC50(nM) |
| 89 | Ex9-39amide, dK7(PEG2-γE(C16)), G8 | 6.2 |
| 90 | Ex9-40amide, K40(C18 diacid) | 28 |
| 91 | Ex9-40amide, W11, K40(C18 diacid) | 43 |
| 92 | Ex9-40amide, dW11, K40(C18 diacid) | 32 |
| 93 | Ex9-40amide, W11, K40(mPeg-γE-C18 diacid) | 87 |
| 94 | Ex9-40amide, dW11, K40(mPeg-γE-C18 diacid) | 115 |

1. A GLP-1 receptor antagonist, said antagonist comprising the amino acid sequence of R₁₀-DVX₁₁X₁₂YLX₁₅X₁₆QAX₁₉X₂₀EFX₂₃X₂₄WLVRGGPSSGAPPPSX₄₀-R₂₀ SEQ ID NO: 97), wherein
R₁₀ is NH₂ or an N-terminal extension of 1 or 2 amino acids, wherein one of the amino acids of the N-terminal extension is acylated with a C14-C20 fatty acid or diacid, optionally via a spacer, optionally wherein R₁₀ is a dipeptide of the structure: X₇X₈-, wherein X₇ is an acylated amino acid, optionally acylated Lys or acylated dLys, and X₈ is Gly or a C₁-C₄ N-alkylated Gly;
X₁₁ is Trp, dTrp or Ser;
X₁₂ is Arg, Ser or an acylated amino acid, optionally acylated Lys;
X₁₅ is Glu or dGlu;
X₁₆ is Glu, dGlu, Asp, homoglutamic acid or homocysteic acid;
X₁₉ is Val, cyclopropane, cyclopentane, cyclohexane or phenyl glycine;
X₂₀ is Arg, homolysine or citrulline;
X₂₃ is Ile, or dile;
X₂₄ is Glu, or Ala; and
X₄₀ is a bond or an acylated amino acid, optionally an acylated Lys; and
R₂₀ is COOH or CONH₂-, optionally wherein 1, 2 or 3 amino acids selected from positions 7, 10, 13, or 16 are substituted with Trp or dTrp, with the proviso that when one of X₁₀ or X₁₁ is Trp or dTrp then the other is not Trp or dTrp and that R₁₀ and X₁₂ cannot both comprise an acylated amino acid; optionally wherein 1, 2 or 3 amino acids at any of positions 16, 18, 19, 24, 26, or 28 are substituted with Aib.

2. The GLP-1 antagonist of claim 1 wherein said antagonist comprises the amino acid sequence of
R₁₀-DVX₁₁X₁₂YLX₁₅X₁₆QAX₁₉X₂₀EFX₂₃EWLVRGGPSSGAPPPSX₄₀-R₂₀ SEQ ID NO: 23), wherein
R₁₀ is NH₂ or X₇X₈-, wherein X₇ is an amino acid acylated with a C14-C20 fatty acid or diacid, optionally via a spacer and X₈ is Gly or a C₁-C₄ N-alkylated Gly;
X₁₁ is Trp, dTrp or Ser;
X₁₂ is Arg, Ser or an acylated amino acid, optionally acylated Lys;
X₁₅ is Glu or dGlu;
X₁₆ is Glu, dGlu, Asp, homoglutamic acid or homocysteic acid;
X₁₉ is Val, cyclopropane, cyclopentane, cyclohexane or phenyl glycine;
X₂₀ is Arg, homolysine or citrulline;
X₂₃ is Ile, or dile; and
X₄₀ is an acylated amino acid, optionally an acylated Lys; and
R₂₀ is COOH or CONH₂-, optionally wherein 1, 2 or 3 amino acids at any of positions 16, 18, 19, 24, 26, or 28 are substituted with Aib.

3. The GLP-1 antagonist of claim 1 wherein:
i) each acylated amino acid of the GLP-1 antagonist is an acylated Lys; or
ii) X₇ is an acylated Lys and X₁₂ is Arg; or
iii) R₁₀ is NH₂ and X₁₂ is an acylated Lys; or
iv) X₇ is NH₂ and X₁₂ is Arg.

4-6. (canceled)

7. The GLP-1 antagonist of claim 1 wherein the antagonist comprises an amino acid sequence selected from the group consisting of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, and SEQ ID NO: 42.

8. The GLP-1 antagonist of claim 1 wherein the acylated amino acids of said GLP-1 antagonist are independently a Lys residue acylated with a C16-C18 fatty acid or C16-C18 fatty diacid directly linked to the Lys side chain or optionally via a spacer comprising a
i) gamma glutamic acid,
ii) minipeg polymer: —[COCH₂(OCH₂CH₂)ₖNH]—, wherein k is 2, 4, 6 or 8,
iii) or any multiplicity or combination of i) or ii).

9. A GLP-1 receptor antagonist, said antagonist comprising the amino acid sequence of
I)
(SEQ ID NO: 98)
DX₁₀X₁₁X₁₂YLX₁₅X₁₆QAVREFX₂₃X₂₄WLVRGGPSSGAPPPS;
wherein
X₁₀ is Trp, dTrp or Val;
X₁₁ is Trp, dTrp or Ser;
X₁₂ is Arg, Lys or Ser;
X₁₅ is Glu or dGlu;
X₁₆ is Trp, dTrp, dGlu or Glu;
X₂₃ is Ile or dIle;
X₂₄ is Ala or Glu;
said GLP-1 receptor antagonist being acylated with a fatty acid or diacid group of sufficient size to bind serum albumin with high affinity, optionally wherein an amino acid of the GLP-1 receptor antagonist is acylated with a C16-C18 fatty acid or C16-C18 fatty diacid; or
II)
(SEQ ID NO: 5)
DX₁₀X₁₁RYLX₁₅X₁₆QAVREFX₂₃EWLVRGGPSSGAPPPSX₄₀R₂₀,
wherein
A)
X₁₀ is Trp, dTrp or Val;
X₁₁ is Trp, dTrp or Ser, optionally wherein X₁₁ is Trp or dTrp;
X₁₅ is Glu or dGlu
X₁₆ is Trp, dTrp, dGlu or Glu;
X₂₃ is Ile or dIle;
X₄₀ is an acylated amino acid; and
R₂₀ is COOH or CONH₂-, optionally wherein the peptide comprises one or more substitutions of Aib at any of positions 16, 18, 19, 24, 26 or 28, or optionally a substitution of an acylated Lys at position 12, wherein said position number is relative to the native Exendin4 amino acid sequence; or
B)
X₁₀ is Trp, dTrp or Val
X₁₁ is Trp or dTrp;
X₁₅ is Glu or dGlu;
X₁₆ is Glu or dGlu;
X₂₃ is Ile or dIle; and
X₄₀ is Lys acylated with a C16 or C18 diacid, optionally wherein the diacid is linked via a spacer comprising the structure:
—[COCH₂(OCH₂CH₂)ₖNH]_{q}-(gamma glutamic acid)ₚ-
wherein k is 2, and p and q are independently an integer selected from 1 or 2, and
R₂₀ is COOH or CONH₂-.

10-11. (canceled)

12. The GLP-1 antagonist of claim 9 wherein an amino acid at position 16, 18, 19, 24, 26 or 28 is substituted with Aib, optionally wherein the amino acid at position 12 is substituted with an acylated Lys.

13. (canceled)

14. The GLP-1 antagonist of claim 9 wherein
i)
X₁₅ is dGlu;
X₁₆ is Glu; and
X₂₃ is Ile; or
ii)
X₁₅ is Glu;
X₁₆ is Glu; and
X₂₃ is Ile.

15-16. (canceled)

17. The GLP-1 antagonist of claim 9 wherein X₄₀ is an amino acid comprising a structure of Formula I (optionally, Lys), Formula II (optionally, Cys), or Formula III (optionally, Ser), wherein each of Formulae I, II, and III, is: 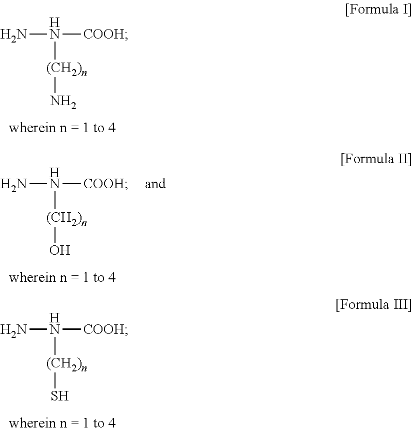
or
X₄₀ is an acylated lysine.

18. (canceled)

19. The GLP-1 antagonist of claim 9 wherein the acyl group of the acylated amino acid is selected from (C₁-C₄ alkyl)NH-CO(CH₂)₁₄₋₂₀CH₃-, (C₁-C₄ alkyl)NH[spacer]-CO(CH₂)₁₄₋₂₀CH₃-, (C₁-C₄ alkyl)NH-CO(CH₂)₁₄₋₂₀COOH or (C₁-C₄ alkyl)NH[spacer]-CO(CH₂)₁₄₋₂₀COOH.

20. The GLP-1 antagonist of claim 9 wherein the acyl group of the acylated amino acid is covalently linked to the amino acid side chain of the acylated amino acid via a spacer, wherein said spacer is
i) an amino acid or dipeptide; or
ii) a gamma glutamic acid, or
iii) a minipeg polymer: —[COCH₂(OCH₂CH₂)ₖNH]—, wherein k is 2, 4, 6 or 8, or
iv) or any multiplicity or combination of ii) and/or iii).

21-22. (canceled)

23. The GLP-1 antagonist of claim 20, wherein the acylated amino acid is linked to a C16 to C18 fatty acid or C16 to C18 fatty diacid, optionally wherein the acid or diacid is linked via a spacer comprising the structure:
-(gamma glutamic acid)ₚ—[COCH₂(OCH₂CH₂)ₖNH]_{q}-(gamma glutamic acid)ₙ- or
—[COCH₂(OCH₂CH₂)ₖNH]_{q}-(gamma glutamic acid)ₚ—[COCH₂(OCH₂CH₂)ₖNH]ₘ-;
wherein k is an integer selected from 2, 4 or 8, m and n are independently an integer selected from 0, 1 or 2, and p and q are independently an integer selected from 1, 2, 4 or 8.

24. The GLP-1 antagonist of claim 23, wherein the acylated amino acid is Lys having a C16 to C18 fatty acid linked to the lysine side chain via a spacer comprising the structure:
—[COCH₂(OCH₂CH₂)ₖNH]_{q}-(gamma glutamic acid)ₚ-
wherein k is 2, and p and q are independently an integer selected from 1 or 2.

25. (canceled)

26. The GLP-1 antagonist of claim 9 wherein R₂₀ is CONH₂-.

27. A derivative of the GLP-1 antagonist of claim 9 further comprising a dipeptide A-B: 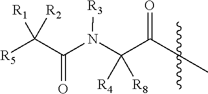
linked to said GLP-1 antagonist through an amide bond wherein
R₁-, R₂-, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃-, (C₁-C₄ alkyl)CONH₂-, (C₁-C₄ alkyl)COOH, (C₁-C₄ alkyl)NH₂-, (C₁-C₄ alkyl)NHC(NH₂⁺)NH₂-, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇-, (C₁-C₄ alkyl)(C₃-C₉ heteroaryl), and C₁-C₁₂ alkyl(W1)C₁-C₁₂ alkyl, wherein W1 is a heteroatom selected from the group consisting of N, S and O;
R₃ is selected from the group consisting of C₁-C₁₈ alkyl, (C₁-C₁₈ alkyl)OH, (C₁-C₁₈ alkyl)NH₂-, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇-, and (C₁-C₄ alkyl)(C₃-C₉ heteroaryl) or R₄ and R₃ together with the atoms to which they are attached form a pyrrolidine ring;
R₅ is NHR₆ or OH;
R₆ is H, C₁-C₈ alkyl; and
R₇ is selected from the group consisting of H and OH
wherein the chemical cleavage half-life (t_{1/2}) of A-B from said GLP-1 antagonist is at least about 1 hour to about 1 week in PBS under physiological conditions.

28. The derivative of claim 27 wherein the dipeptide A-B is covalently linked to the N-terminal alpha amine of the GLP-1 antagonist amino acid sequence.

29. The derivative of claim 27 wherein
I)
R₁ and R₈ are independently H or C₁-C₈ alkyl;
R₂ and R₄ are independently selected from the group consisting of H, C₁-C₈ alkyl, (C₁-C₄ alkyl)OH, (C₁-C₄ alkyl)SH, (C₂-C₃ alkyl)SCH₃-, (C₁-C₄ alkyl)CONH₂-, (C₁-C₄ alkyl)COOH, (C₁-C₄ alkyl)NH₂-, and (C₁-C₄ alkyl)(C₆ aryl)R₇-;
R₃ is C₁-C₆ alkyl;
R₅ is NH_{2;} and
R₇ is selected from the group consisting of hydrogen, and OH; or
II)
R₁-, comprises a side chain selected from the group consisting of C₁-C₈ alkyl, (C₁-C₄ alkyl)OH, (C₁-C₄ alkyl)SH, (C₁-C₄ alkyl)COOH, and (C₁-C₄ alkyl)NH₂-, optionally wherein a C16-C30 fatty acid or C16-C30 diacid is covalently linked to said side chain, optionally via a spacer selected from the group consisting of a gamma glutamic acid, a gamma glutamic acid-gamma glutamic acid dipeptide, and a gamma glutamic acid-[COCH₂(OCH₂CH₂)ₖNH]_{q}-gamma glutamic acid, wherein
k is an integer selected from the range of 1-8; and
q is an integer selected from the range of 1-8, optionally wherein k is 2 and q is selected from the range of 1-8;
R₂-, R₄ and R₈ are independently H, or C₁-C₄ alkyl;
R₃ is C₁-C₆ alkyl; and
R₅ is NH₂-, or
III)
R₁ is H, C₁-C₄ alkyl, (C₁-C₄ alkyl)OH or (C₁-C₄ alkyl)NH₂-;
R₂ is H,
R₃ is C₁-C₄ alkyl;
R₄ is H, or C₁-C₄ alkyl;
R₅ is NH₂-; and
R₈ is hydrogen, or
IV)
R₁ is (C₄ alkyl)NH₂ or (C₄ alkyl)NH(mPeg-γE-diacid)-C18;
R₂-, R₄ and R₈ are each H;
R₃ is C₁-C₄ alkyl; and
R₅ is NH₂-;
R₅ is an amine.

30-31. (canceled)

32. The derivative of claim 27, wherein said dipeptide A-B comprises an acylated Lys residue and an N-alkylated Gly residue, wherein said Lys and N-alkylated Gly residues are linked via a peptide bond, optionally wherein said Lys residue is in the D-conformation.

33. (canceled)

34. A pharmaceutical composition comprising a GLP-1 antagonist of claim 1, and a pharmaceutically acceptable carrier, diluent, or excipient.

35. A method of treating a patient suffering from atypical hypoglycemia said method comprising the step of administering to a patient in need thereof a pharmaceutical composition of claim 34 in an amount effective to elevate blood glucose levels.


SEQUENCE LISTING (98 sequences)

SEQ ID NO: 1 - protein, 39 aa, Heloderma suspectum
HGEGTFTSDVSKQMEEEAVRLFIEWLKNGGPSSGAPPPS

SEQ ID NO: 2 - protein, 32 aa, Heloderma suspectum
SDVSKQMEEEAVRLFIEWLKNGGPSSGAPPPS

SEQ ID NO: 3 - protein, 32 aa, Artificial Sequence
  Analog of Exendin 4
  MOD_RES (32)..(32): acylation with C16 fatty acid
DVSSYLEEQAVREFIAWLVKGGPSSGAPPPSK

SEQ ID NO: 4 - protein, 32 aa, Artificial Sequence
  Analog of Exendin 4
DVSRYLEEQAVREFIEWLVRGGPSSGAPPPSK

SEQ ID NO: 5 - protein, 32 aa, Artificial Sequence
  Analog of Exendin 4
  MISC_FEATURE (2)..(2): Xaa at position 2 is Trp, dTrp or Val
  MISC_FEATURE (3)..(3): Xaa at position 3 is Trp, dTrp or Ser
  MISC_FEATURE (7)..(7): Xaa at position 7 is Glu or dGlu
  MISC_FEATURE (8)..(8): Xaa at position 8 is Trp, dTrp, Glu or dGlu
  MISC_FEATURE (15)..(15): Xaa at position 15 is Ile or dIle
  MISC_FEATURE (32)..(32): Xaa at position 32 is any acylated amino acid
DXXRYLXXQAVREFXEWLVRGGPSSGAPPPSX

SEQ ID NO: 6 - protein, 32 aa, Artificial Sequence
  Analog of Exendin 4
  MISC_FEATURE (3)..(3): Xaa at position 3 is Trp or dTrp
  MISC_FEATURE (32)..(32): Xaa at position 32 is an acylated amino acid
DVXRYLEEQAVREFIEWLVRGGPSSGAPPPSX

SEQ ID NO: 7 - protein, 32 aa, Artificial Sequence
  Analog of Exendin 4
  MISC_FEATURE (32)..(32): Xaa at position 32 is an acylated amino acid
DVWRYLEEQAVREFIEWLVRGGPSSGAPPPSX

SEQ ID NO: 8 - protein, 32 aa, Artificial Sequence
  Analog of Exendin 4
  MISC_FEATURE (4)..(4): Xaa at position 4 is an acylated Lys
  MISC_FEATURE (32)..(32): Xaa at position 32 is an acyltated Lys
  MOD_RES (32)..(32): AMIDATION
DVWXYLEEQAVREFIEWLVRGGPSSGAPPPSX

SEQ ID NO: 9 - protein, 32 aa, Artificial Sequence; identical to SEQ ID NO: 6
  Analog of Exendin 4
  MISC_FEATURE (3)..(3): Xaa at position 3 is Trp or dTrp
  MISC_FEATURE (32)..(32): Xaa at position 32 is an acylated amino acid

SEQ ID NO: 10 - protein, 32 aa, Artificial Sequence; identical to SEQ ID NO: 6
  Analog of Exendin 4
  MISC_FEATURE (3)..(3): Xaa at position 3 is Trp or dTrp
  MISC_FEATURE (32)..(32): Xaa at position 32 is an acylated amino acid
  MOD_RES (32)..(32): AMIDATION

SEQ ID NO: 11 - protein, 32 aa, Artificial Sequence
  Analog of Exendin 4
  misc_feature (3)..(3): Xaa at position 3 is Trp or dTrp
  misc_feature (7)..(7): Xaa at position 7 is Glu or dGlu
  misc_feature (8)..(8): Xaa at position 8 is Glu or dGlu
  misc_feature (15)..(15): Xaa at position 15 is Ile or dIle
DVXRYLXXQAVREFIEWLVRGGPSSGAPPPSK

SEQ ID NO: 12 - protein, 32 aa, Artificial Sequence
  Analog of Exendin 4
  MISC_FEATURE (32)..(32): Xaa at position 32 is Lys acylated with (miniPEG)2- gamma Glu-C16
DVSSYLEEQAVREFIAWLVKGGPSSGAPPPSX

SEQ ID NO: 13 - protein, 32 aa, Artificial Sequence
  Analog of Exendin 4
  MISC_FEATURE (32)..(32): Xaa at position 32 is Lys acylated with C16 fatty acid
DVSRYLEEQAVREFIEWLVRGGPSSGAPPPSX

SEQ ID NO: 14 - protein, 34 aa, Artificial Sequence
  Analog of Exendin 4
  MISC_FEATURE (1)..(1): Xaa at position 1 is dLys acylated with (mPeg-gammaGlu-diacid C18)
  MISC_FEATURE (2)..(2): Xaa at position 1 is N-Methyl-Gly
  MISC_FEATURE (34)..(34): Xaa at position 34 is Lys acylated with C16 fatty acid
XXDVSRYLEEQAVREFIEWLVRGGPSSGAPPPSX

SEQ ID NO: 15 - protein, 34 aa, Artificial Sequence; identical to SEQ ID NO: 14
  Analog of Exendin 4
  MISC_FEATURE (1)..(1): Xaa at position 1 is dLys acylated with (mPeg-gammaGlu-diacid C18)
  MISC_FEATURE (2)..(2): Xaa at position 2 is N-isopropylr-Gly
  MISC_FEATURE (34)..(34): Xaa at position 34 is Lys acylated with C16 fatty acid

SEQ ID NO: 16 - protein, 32 aa, Artificial Sequence; identical to SEQ ID NO: 13
  Analog of Exendin 4
  MISC_FEATURE (32)..(32): Xaa at position 32 is Lys acylated with mPEG-gammaGlu-C16

SEQ ID NO: 17 - protein, 34 aa, Artificial Sequence
  Analog of Exendin 4
  MISC_FEATURE (1)..(1): Xaa at position 1 is dLys acylated with (mPeg-gammaGlu-diacid C18)
  MISC_FEATURE (34)..(34): Xaa at position 34 is Lys acylated with mPeg-gammaGlu-C16 fatty acid
XGDVSRYLEEQAVREFIEWLVRGGPSSGAPPPSX

SEQ ID NO: 18 - protein, 34 aa, Artificial Sequence; identical to SEQ ID NO: 14
  Analog of Exendin 4
  MISC_FEATURE (1)..(1): Xaa at position 1 is dLys acylated with (mPeg-gammaGlu-diacid C18 diacid)
  MISC_FEATURE (2)..(2): Xaa at position 2 is N-isopropyl Gly
  MISC_FEATURE (34)..(34): Xaa at position 34 is Lys acylated with mPeg-gammaGlu-C16 fatty acid

SEQ ID NO: 19 - protein, 34 aa, Artificial Sequence; identical to SEQ ID NO: 14
  Analog of Exendin 4
  MISC_FEATURE (1)..(1): Xaa at position 1 is dLys acylated with (mPeg-gammaGlu-diacid C18 diacid)
  MISC_FEATURE (2)..(2): Xaa at position 2 is N-Methyl-Gly
  MISC_FEATURE (34)..(34): Xaa at position 32 is Lys acylated withmPeg- gammaGlu-C16 fatty acid

SEQ ID NO: 20 - protein, 34 aa, Artificial Sequence; identical to SEQ ID NO: 17
  Analog of Exendin 4
  MISC_FEATURE (1)..(1): Xaa at position 1 is dLys acylated with (mPeg-gammaGlu-diacid C18)2
  MISC_FEATURE (34)..(34): Xaa at position 34 is Lys acylated with mPeg- gammaGlu-C18 diacid

SEQ ID NO: 21 - protein, 34 aa, Artificial Sequence; identical to SEQ ID NO: 17
  Analog of Exendin 4
  MISC_FEATURE (1)..(1): Xaa at position 1 is dLys acylated with (mPeg-gammaGlu-diacid C18)2
  MISC_FEATURE (34)..(34): Xaa at position 34 is Lys acylated with mPeg-gammaGlu-C16 fatty acid

SEQ ID NO: 22 - protein, 34 aa, Artificial Sequence; identical to SEQ ID NO: 14
  Analog of Exendin 4
  MISC_FEATURE (1)..(1): Xaa at position 1 is dLys acylated with (mPeg-gammaGlu-diacid C18)
  MISC_FEATURE (2)..(2): Xaa at position 2 is N-Methyl Gly
  MISC_FEATURE (34)..(34): Xaa at position 34 is Lys acylated with mPeg-gammaGlu-C16 fatty acid

SEQ ID NO: 23 - protein, 32 aa, Artificial Sequence
  Analog of Exendin 4
  MISC_FEATURE (3)..(3): Xaa at position 3 is Trp, dTrp or Ser
  MISC_FEATURE (4)..(4): Xaa at position 4 is Arg or an acylated amino acid
  MISC_FEATURE (7)..(7): Xaa at position 7 is Glu or dGlu
  MISC_FEATURE (8)..(8): Xaa at position 8 is Glu, dGlu, Asp, homoglutamic acid or homocysteic acid;
  MISC_FEATURE (11)..(11): Xaa at position 11 is Val, cyclopropane, cyclopentane, cyclohexane or phenyl glysine
  MISC_FEATURE (12)..(12): Xaa at position 12 is Arg, homolysine or citrulline
  MISC_FEATURE (15)..(15): Xaa at position 15 is Ile or dIle
  MISC_FEATURE (32)..(32): Xaa at position 32 is any acylated amino acid
DVXXYLXXQAXXEFXEWLVRGGPSSGAPPPSX

SEQ ID NO: 24 - protein, 32 aa, Artificial Sequence
  Analog of Exendin 4
  MISC_FEATURE (3)..(3): Xaa at position 3 is Trp, dTrp or Ser
  MISC_FEATURE (4)..(4): Xaa at position 4 is Arg or an acylated amino acid
  MISC_FEATURE (8)..(8): Xaa at position 8 is Glu or Asp
  MISC_FEATURE (32)..(32): Xaa at position 32 is any acylated amino acid
DVXXYLEXQAVREFIEWLVRGGPSSGAPPPSX

SEQ ID NO: 25 - protein, 32 aa, Artificial Sequence
  Analog of Exendin 4
  MISC_FEATURE (3)..(3): Xaa at position 3 is Trp, dTrp or Ser
  MISC_FEATURE (7)..(7): Xaa at position 7 is Glu or dGlu
  MISC_FEATURE (8)..(8): Xaa at position 8 is Glu or dGlu
  misc_feature (9)..(9): Xaa can be any naturally occurring amino acid
  MISC_FEATURE (15)..(15): Xaa at position 15 is Ile or dIle
  MISC_FEATURE (32)..(32): Xaa at position 32 is any acylated amino acid
DVXRYLQXXAVREFXEWLVRGGPSSGAPPPSX

SEQ ID NO: 26 - protein, 34 aa, Artificial Sequence
  Analog of Exendin 4
  MISC_FEATURE (1)..(1): Xaa at position 1 is an acylated Lys
  MISC_FEATURE (2)..(2): Xaa at position 2 is Gly or Sarcosine
  MISC_FEATURE (5)..(5): Xaa at position 5 is Trp or dTrp
  MISC_FEATURE (34)..(34): Xaa at position 34 is an acylated amino acid
XXDVXRYLEEQAVREFIEWLVRGGPSSGAPPPSX

SEQ ID NO: 27 - protein, 32 aa, Artificial Sequence; identical to SEQ ID NO: 6
  Analog of Exendin 4
  MISC_FEATURE (3)..(3): Xaa at position 3 is dTrp
  MISC_FEATURE (32)..(32): Xaa at position 32 is an acylated amino acid

SEQ ID NO: 28 - protein, 32 aa, Artificial Sequence; identical to SEQ ID NO: 7
  Analog of Exendin 4
  MISC_FEATURE (32)..(32): Xaa at position 32 is an acylated amino acid

SEQ ID NO: 29 - protein, 32 aa, Artificial Sequence
  Analog of Exendin 4
  MISC_FEATURE (3)..(3): Xaa at position 3 is dTrp
  MISC_FEATURE (8)..(8): Xaa at position 8 is Aib
  MISC_FEATURE (32)..(32): Xaa at position 32 is an acylated amino acid
DVXRYLEXQAVREFIEWLVRGGPSSGAPPPSX

SEQ ID NO: 30 - protein, 32 aa, Artificial Sequence
  Analog of Exendin 4
  MISC_FEATURE (8)..(8): Xaa at position 8 is Aib
  MISC_FEATURE (32)..(32): Xaa at position 32 is an acylated amino acid
DVWRYLEXQAVREFIEWLVRGGPSSGAPPPSX

SEQ ID NO: 31 - protein, 32 aa, Artificial Sequence
  Analog of Exendin 4
  MISC_FEATURE (3)..(3): Xaa at position 3 is dTrp
  MISC_FEATURE (10)..(10): Xaa at position 10 is Aib
  MISC_FEATURE (32)..(32): Xaa at position 32 is an acylated amino acid
DVXRYLEEQXVREFIEWLVRGGPSSGAPPPSX

SEQ ID NO: 32 - protein, 32 aa, Artificial Sequence
  Analog of Exendin 4
  MISC_FEATURE (10)..(10): Xaa at position 10 is Aib
  MISC_FEATURE (32)..(32): Xaa at position 32 is an acylated amino acid
DVWRYLEEQXVREFIEWLVRGGPSSGAPPPSX

SEQ ID NO: 33 - protein, 32 aa, Artificial Sequence
  Analog of Exendin 4
  MISC_FEATURE (3)..(3): Xaa at position 3 is dTrp
  MISC_FEATURE (11)..(11): Xaa at position 11 is Aib
  MISC_FEATURE (32)..(32): Xaa at position 32 is an acylated amino acid
DVXRYLEEQAXREFIEWLVRGGPSSGAPPPSX

SEQ ID NO: 34 - protein, 32 aa, Artificial Sequence
  Analog of Exendin 4
  MISC_FEATURE (11)..(11): Xaa at position 11 is Aib
  MISC_FEATURE (32)..(32): Xaa at position 32 is an acylated amino acid
DVWRYLEEQAXREFIEWLVRGGPSSGAPPPSX

SEQ ID NO: 35 - protein, 32 aa, Artificial Sequence
  Analog of Exendin 4
  MISC_FEATURE (3)..(3): Xaa at position 3 is dTrp
  MISC_FEATURE (16)..(16): Xaa at position 16 is Aib
  MISC_FEATURE (32)..(32): Xaa at position 32 is an acylated amino acid
DVXRYLEEQAVREFIXWLVRGGPSSGAPPPSX

SEQ ID NO: 36 - protein, 32 aa, Artificial Sequence
  Analog of Exendin 4
  MISC_FEATURE (16)..(16): Xaa at position 16 is Aib
  MISC_FEATURE (32)..(32): Xaa at position 32 is an acylated amino acid
DVWRYLEEQAVREFIXWLVRGGPSSGAPPPSX

SEQ ID NO: 37 - protein, 32 aa, Artificial Sequence
  Analog of Exendin 4
  MISC_FEATURE (3)..(3): Xaa at position 3 is dTrp
  MISC_FEATURE (18)..(18): Xaa at position 18 is Aib
  MISC_FEATURE (32)..(32): Xaa at position 32 is an acylated amino acid
DVXRYLEEQAVREFIEWXVRGGPSSGAPPPSX

SEQ ID NO: 38 - protein, 32 aa, Artificial Sequence
  Analog of Exendin 4
  MISC_FEATURE (18)..(18): Xaa at position 18 is Aib
  MISC_FEATURE (32)..(32): Xaa at position 32 is an acylated amino acid
DVWRYLEEQAVREFIEWXVRGGPSSGAPPPSX

SEQ ID NO: 39 - protein, 32 aa, Artificial Sequence
  Analog of Exendin 4
  MISC_FEATURE (3)..(3): Xaa at position 3 is dTrp
  MISC_FEATURE (20)..(20): Xaa at position 20 is Aib
  MISC_FEATURE (32)..(32): Xaa at position 32 is an acylated amino acid
DVXRYLEEQAVREFIEWLVXGGPSSGAPPPSX

SEQ ID NO: 40 - protein, 32 aa, Artificial Sequence
  Analog of Exendin 4
  MISC_FEATURE (20)..(20): Xaa at position 20 is Aib
  MISC_FEATURE (32)..(32): Xaa at position 32 is an acylated amino acid
DVWRYLEEQAVREFIEWLVXGGPSSGAPPPSX

SEQ ID NO: 41 - protein, 32 aa, Artificial Sequence
  Analog of Exendin 4
  MISC_FEATURE (3)..(3): Xaa at position 3 is dTrp
  MISC_FEATURE (12)..(12): Xaa at position 12 is dArg
  MISC_FEATURE (32)..(32): Xaa at position 32 is an acylated amino acid
DVXRYLEEQAVXEFIEWLVRGGPSSGAPPPSX

SEQ ID NO: 42 - protein, 32 aa, Artificial Sequence
  Analog of Exendin 4
  MISC_FEATURE (12)..(12): Xaa at position 12 is dArg
  MISC_FEATURE (32)..(32): Xaa at position 32 is an acylated amino acid
DVWRYLEEQAVXEFIEWLVRGGPSSGAPPPSX

SEQ ID NO: 43 - protein, 32 aa, Artificial Sequence
  Analog of Exendin 4
  MISC_FEATURE (20)..(20): Xaa at position 20 is Aib
  MOD_RES (32)..(32): acylation with C16 fatty acid
  MOD_RES (32)..(32): AMIDATION
DVSSYLEEQAVREFIAWLKXGGPSSGAPPPSK

SEQ ID NO: 44 - protein, 32 aa, Artificial Sequence
  Analog of Exendin 4
  MISC_FEATURE (18)..(18): Xaa at position 18 is Aib
  MOD_RES (32)..(32): acylation with C16 fatty acid
  MOD_RES (32)..(32): AMIDATION
DVSSYLEEQAVREFIAWXVKGGPSSGAPPPSK

SEQ ID NO: 45 - protein, 32 aa, Artificial Sequence; identical to SEQ ID NO: 3
  Analog of Exendin 4
  MISC_FEATURE (11)..(11): Xaa at position 11 is Aib
  MOD_RES (32)..(32): acylation with C16 fatty acid
  MOD_RES (32)..(32): AMIDATION

SEQ ID NO: 46 - protein, 32 aa, Artificial Sequence; identical to SEQ ID NO: 3
  Analog of Exendin 4
  MISC_FEATURE (10)..(10): Xaa at position 10 is Aib
  MOD_RES (32)..(32): acylation with C16 fatty acid
  MOD_RES (32)..(32): AMIDATION

SEQ ID NO: 47 - protein, 32 aa, Artificial Sequence; identical to SEQ ID NO: 3
  Analog of Exendin 4
  MOD_RES (32)..(32): acylation with C16 fatty acid
  MOD_RES (32)..(32): AMIDATION

SEQ ID NO: 48 - protein, 32 aa, Artificial Sequence; identical to SEQ ID NO: 3
  Analog of Exendin 4
  MOD_RES (32)..(32): acylation with [(miniPEG)2-gE-C16]
  MOD_RES (32)..(32): AMIDATION

SEQ ID NO: 49 - protein, 32 aa, Artificial Sequence
  Analog of Exendin 4
  MISC_FEATURE (7)..(7): Xaa at position 7 is dGlu
  MOD_RES (32)..(32): acylation with C16 fatty acid
  MOD_RES (32)..(32): AMIDATION
DVSSYLXEQAVREFIAWLVKGGPSSGAPPPSK

SEQ ID NO: 50 - protein, 32 aa, Artificial Sequence
  Analog of Exendin 4
  MISC_FEATURE (11)..(11): Xaa at position 11 is dVal
  MOD_RES (32)..(32): acylation with C16 fatty acid
  MOD_RES (32)..(32): AMIDATION
DVSSYLEEQAXREFIAWLVKGGPSSGAPPPSK

SEQ ID NO: 51 - protein, 32 aa, Artificial Sequence
  Analog of Exendin 4
  MISC_FEATURE (15)..(15): Xaa at position 15 is dIle
  MOD_RES (32)..(32): acylation with (C16]
  MOD_RES (32)..(32): AMIDATION
DVSSYLEEQAVREFXAWLVKGGPSSGAPPPSK

SEQ ID NO: 52 - protein, 32 aa, Artificial Sequence; identical to SEQ ID NO: 3
  Analog of Exendin 4
  MOD_RES (32)..(32): acylation with [(gE)-C16]
  MOD_RES (32)..(32): AMIDATION

SEQ ID NO: 53 - protein, 32 aa, Artificial Sequence; identical to SEQ ID NO: 3
  Analog of Exendin 4
  MOD_RES (32)..(32): acylation with [(gE)2-C16]
  MOD_RES (32)..(32): AMIDATION

SEQ ID NO: 54 - protein, 32 aa, Artificial Sequence; identical to SEQ ID NO: 3
  Analog of Exendin 4
  MOD_RES (32)..(32): acylation with [(gE)4-C16]
  MOD_RES (32)..(32): AMIDATION

SEQ ID NO: 55 - protein, 32 aa, Artificial Sequence; identical to SEQ ID NO: 3
  Analog of Exendin 4
  MOD_RES (32)..(32): acylation with [(gE)6-C16]
  MOD_RES (32)..(32): AMIDATION

SEQ ID NO: 56 - protein, 32 aa, Artificial Sequence; identical to SEQ ID NO: 3
  Analog of Exendin 4
  MOD_RES (32)..(32): acylation with [(miniPEG)-gE-C16]
  MOD_RES (32)..(32): AMIDATION

SEQ ID NO: 57 - protein, 32 aa, Artificial Sequence; identical to SEQ ID NO: 3
  Analog of Exendin 4
  MOD_RES (32)..(32): acylation with [gE-(miniPEG)2-gE-C16]
  MOD_RES (32)..(32): AMIDATION

SEQ ID NO: 58 - protein, 32 aa, Artificial Sequence; identical to SEQ ID NO: 3
  Analog of Exendin 4
  MOD_RES (32)..(32): acylation with [(miniPEG)2-(gE)2-C16]
  MOD_RES (32)..(32): AMIDATION

SEQ ID NO: 59 - protein, 32 aa, Artificial Sequence; identical to SEQ ID NO: 3
  Analog of Exendin 4
  MOD_RES (32)..(32): acylation with [(miniPEG)-(gE)-(miniPEG)-(gE)- C16]
  MOD_RES (32)..(32): AMIDATION

SEQ ID NO: 60 - protein, 32 aa, Artificial Sequence; identical to SEQ ID NO: 3
  Analog of Exendin 4
  MOD_RES (32)..(32): acylation with [(miniPEG)-(gE)2-(miniPEG)-C16]
  MOD_RES (32)..(32): AMIDATION

SEQ ID NO: 61 - protein, 32 aa, Artificial Sequence; identical to SEQ ID NO: 3
  Analog of Exendin 4
  MOD_RES (32)..(32): acylation with [(gE)2-(miniPEG)2-C16]
  MOD_RES (32)..(32): AMIDATION

SEQ ID NO: 62 - protein, 32 aa, Artificial Sequence; identical to SEQ ID NO: 3
  Analog of Exendin 4
  MOD_RES (32)..(32): acylation with [(gE)-(miniPEG)-(gE)-(miniPEG)- C16]
  MOD_RES (32)..(32): AMIDATION

SEQ ID NO: 63 - protein, 32 aa, Artificial Sequence; identical to SEQ ID NO: 3
  Analog of Exendin 4
  MOD_RES (32)..(32): acylation with [diacidC18]
  MOD_RES (32)..(32): AMIDATION

SEQ ID NO: 64 - protein, 32 aa, Artificial Sequence; identical to SEQ ID NO: 3
  Analog of Exendin 4
  MOD_RES (32)..(32): acylation with [(gE)2-diacid-C18]
  MOD_RES (32)..(32): AMIDATION

SEQ ID NO: 65 - protein, 32 aa, Artificial Sequence; identical to SEQ ID NO: 3
  Analog of Exendin 4
  MOD_RES (32)..(32): acylation with [(gE)4-diacidC18]
  MOD_RES (32)..(32): AMIDATION

SEQ ID NO: 66 - protein, 32 aa, Artificial Sequence; identical to SEQ ID NO: 3
  Analog of Exendin 4
  MOD_RES (32)..(32): acylation with [(gE)6-diacidC18]
  MOD_RES (32)..(32): AMIDATION

SEQ ID NO: 67 - protein, 32 aa, Artificial Sequence; identical to SEQ ID NO: 3
  Analog of Exendin 4
  MOD_RES (32)..(32): acylation with [(miniPEG)2-(gE)diacidC18]
  MOD_RES (32)..(32): AMIDATION

SEQ ID NO: 68 - protein, 32 aa, Artificial Sequence; identical to SEQ ID NO: 3
  Analog of Exendin 4
  MOD_RES (32)..(32): acylation with [(miniPEG)-(gE)-diacidC18]
  MOD_RES (32)..(32): AMIDATION

SEQ ID NO: 69 - protein, 32 aa, Artificial Sequence
  Analog of Exendin 4
  MOD_RES (32)..(32): acylation with C16
  MOD_RES (32)..(32): AMIDATION
DVSSYLEEQAVREFIAWLVRGGPSSGAPPPSK

SEQ ID NO: 70 - protein, 32 aa, Artificial Sequence
  Analog of Exendin 4
  MOD_RES (32)..(32): acylation with C16
  MOD_RES (32)..(32): AMIDATION
DVSSYLEEQAVREFIEWLVRGGPSSGAPPPSK

SEQ ID NO: 71 - protein, 32 aa, Artificial Sequence
  Analog of Exendin 4
  MOD_RES (32)..(32): acylation with C16
  MOD_RES (32)..(32): AMIDATION
DVSSYLEEEAVREFIAWLVRGGPSSGAPPPSK

SEQ ID NO: 72 - protein, 32 aa, Artificial Sequence
  Analog of Exendin 4
  MOD_RES (32)..(32): acylation with C16
  MOD_RES (32)..(32): AMIDATION
DVSSYLEEEAVREFIEWLVRGGPSSGAPPPSK

SEQ ID NO: 73 - protein, 32 aa, Artificial Sequence
  Analog of Exendin 4
  MOD_RES (32)..(32): acylation with C16
  MOD_RES (32)..(32): AMIDATION
DVSRYLEEQAVREFIAWLVRGGPSSGAPPPSK

SEQ ID NO: 74 - protein, 32 aa, Artificial Sequence; identical to SEQ ID NO: 4
  Analog of Exendin 4
  MOD_RES (32)..(32): acylation with C16
  MOD_RES (32)..(32): AMIDATION

SEQ ID NO: 75 - protein, 32 aa, Artificial Sequence
  Analog of Exendin 4
  MOD_RES (32)..(32): acylation with C16
  MOD_RES (32)..(32): AMIDATION
DVSRYLEEEAVREFIEWLVRGGPSSGAPPPSK

SEQ ID NO: 76 - protein, 32 aa, Artificial Sequence
  Analog of Exendin 4
  MOD_RES (32)..(32): acylation with C16
  MOD_RES (32)..(32): AMIDATION
DLSKQMEEEAVRLFIEWLKNGGPSSGAPPPSK

SEQ ID NO: 77 - protein, 32 aa, Artificial Sequence; identical to SEQ ID NO: 69
  Analog of Exendin 4
  MOD_RES (32)..(32): acylation with [miniPEG-gE-C16]

SEQ ID NO: 78 - protein, 32 aa, Artificial Sequence; identical to SEQ ID NO: 70
  Analog of Exendin 4
  MOD_RES (32)..(32): acylation with [miniPEG-gE-C16]

SEQ ID NO: 79 - protein, 32 aa, Artificial Sequence
  Analog of Exendin 4
  MOD_RES (32)..(32): acylation with [miniPEG-gE-C16]
DVSSYLEEQAVREFIEWLRVGGPSSGAPPPSK

SEQ ID NO: 80 - protein, 32 aa, Artificial Sequence
  Analog of Exendin 4
  MISC_FEATURE (20)..(20): Xaa at position 20 is Aib
  MOD_RES (32)..(32): acylation with [miniPEG-gE-C16]
DVSSYLEEQAVREFIEWLRXGGPSSGAPPPSK

SEQ ID NO: 81 - protein, 32 aa, Artificial Sequence
  Analog of Exendin 4
  MISC_FEATURE (19)..(19): Xaa at position 19 is Aib
  MOD_RES (32)..(32): AMIDATION
  MOD_RES (32)..(32): acylation with C16]
DVSSYLEEQAVREFIAWLXKGGPSSGAPPPSK

SEQ ID NO: 82 - protein, 32 aa, Artificial Sequence
  Analog of Exendin 4
  MISC_FEATURE (20)..(20): Xaa at position 20 is Aib
  MOD_RES (32)..(32): AMIDATION
  MOD_RES (32)..(32): acylation with C16]
DVSSYLEEQAVREFIAWKVXGGPSSGAPPPSK

SEQ ID NO: 83 - protein, 32 aa, Artificial Sequence
  Analog of Exendin 4
  MISC_FEATURE (16)..(16): Xaa at position 16 is Aib
  MOD_RES (32)..(32): AMIDATION
  MOD_RES (32)..(32): acylation with C16]
DVSSYLEEQAVREFIXWLVKGGPSSGAPPPSK

SEQ ID NO: 84 - protein, 32 aa, Artificial Sequence
  Analog of Exendin 4
  MISC_FEATURE (8)..(8): Xaa at position 19 is Aib
  MOD_RES (32)..(32): AMIDATION
  MOD_RES (32)..(32): acylation with C16]
DVSSYLEXQAVREFIAWLVKGGPSSGAPPPSK

SEQ ID NO: 85 - protein, 32 aa, Artificial Sequence; identical to SEQ ID NO: 84
  Analog of Exendin 4
  MISC_FEATURE (8)..(8): Xaa at position 8 is dGlu
  MOD_RES (32)..(32): AMIDATION
  MOD_RES (32)..(32): acylation with C16]

SEQ ID NO: 86 - protein, 32 aa, Artificial Sequence
  Analog of Exendin 4
  MISC_FEATURE (9)..(9): Xaa at position 9 is dGln
  MOD_RES (32)..(32): AMIDATION
  MOD_RES (32)..(32): acylation with C16]
DVSSYLEEXAVREFIAWLVKGGPSSGAPPPSK

SEQ ID NO: 87 - protein, 32 aa, Artificial Sequence
  Analog of Exendin 4
  MISC_FEATURE (12)..(12): Xaa at position 12 is dArg
  MOD_RES (32)..(32): AMIDATION
  MOD_RES (32)..(32): acylation with C16]
DVSSYLEEQAVXEFIAWLVKGGPSSGAPPPSK

SEQ ID NO: 88 - protein, 32 aa, Artificial Sequence
  Analog of Exendin 4
  MISC_FEATURE (13)..(13): Xaa at position 13 is dGlu
  MOD_RES (32)..(32): AMIDATION
  MOD_RES (32)..(32): acylation with C16]
DVSSYLEEQAVRXFIAWLVKGGPSSGAPPPSK

SEQ ID NO: 89 - protein, 33 aa, Artificial Sequence
  Analog of Exendin 4
  MISC_FEATURE (1)..(1): Xaa at position 1 is dLys acylated with PEG2-gammaGlu(C16)
  MOD_RES (33)..(33): AMIDATION
XGDLSKQMEEEAVRLFIEWLKNGGPSSGAPPPS

SEQ ID NO: 90 - protein, 32 aa, Artificial Sequence; identical to SEQ ID NO: 76
  Analog of Exendin 4
  MOD_RES (32)..(32): acylation with (C18 diacid)
  MOD_RES (32)..(32): AMIDATION

SEQ ID NO: 91 - protein, 32 aa, Artificial Sequence
  Analog of Exendin 4
  MOD_RES (32)..(32): acylation with (C18 diacid)
  MOD_RES (32)..(32): AMIDATION
DLWKQMEEEAVRLFIEWLKNGGPSSGAPPPSK

SEQ ID NO: 92 - protein, 32 aa, Artificial Sequence
  Analog of Exendin 4
  MISC_FEATURE (3)..(3): Xaa at position 3 is dTrp
  MOD_RES (32)..(32): acylation with (C18 diacid)
  MOD_RES (32)..(32): AMIDATION
DLXKQMEEEAVRLFIEWLKNGGPSSGAPPPSK

SEQ ID NO: 93 - protein, 32 aa, Artificial Sequence; identical to SEQ ID NO: 91
  Analog of Exendin 4
  MOD_RES (32)..(32): acylation with (mPeg-gE-C18 diacid)
  MOD_RES (32)..(32): AMIDATION

SEQ ID NO: 94 - protein, 32 aa, Artificial Sequence; identical to SEQ ID NO: 92
  Analog of Exendin 4
  MISC_FEATURE (3)..(3): Xaa at position 3 is dTrp
  MOD_RES (32)..(32): acylation with (mPeg-gE-C18 diacid)
  MOD_RES (32)..(32): AMIDATION

SEQ ID NO: 95 - protein, 32 aa, Artificial Sequence; identical to SEQ ID NO: 3
  Analog of Exendin 4
  MOD_RES (32)..(32): AMIDATION

SEQ ID NO: 96 - protein, 32 aa, Artificial Sequence
  Analog of Exendin 4
  MISC_FEATURE (7)..(7): Xaa at position 7 is dGlu
  MISC_FEATURE (32)..(32): Xaa at position 32 is an acyltated Lys
  MOD_RES (32)..(32): AMIDATION
DVWRYLXEQAVREFIEWLVRGGPSSGAPPPSX

SEQ ID NO: 97 - protein, 32 aa, Artificial Sequence
  Analog of Exendin 4
  MISC_FEATURE (3)..(3): Xaa at position 3 is Trp, dTrp or Ser
  MISC_FEATURE (4)..(4): Xaa at position 4 is Arg, Ser or an acylated amino acid, optionally acylated Lys
  MISC_FEATURE (7)..(7): Xaa at position 7 is Glu or dGlu
  MISC_FEATURE (8)..(8): Xaa at position 8 is Glu, dGlu, Asp, homoglutamic acid or homocysteic acid
  MISC_FEATURE (11)..(11): Xaa at position 11 is Val, cyclopropane, cyclopentane, cyclohexane or phenyl glycine
  MISC_FEATURE (12)..(12): Xaa at position 12 is Arg, homolysine or citrulline
  MISC_FEATURE (15)..(15): Xaa at position 15 is Ile, or dIle
  MISC_FEATURE (16)..(16): Xaa at position 16 is Glu, or Ala
  MISC_FEATURE (32)..(32): Xaa at position 32 is absent or an acylated amino acid, optionally an acylated Lys
DVXXYLXXQAXXEFXXWLVRGGPSSGAPPPSX

SEQ ID NO: 98 - protein, 31 aa, Artificial Sequence
  Analog of Exendin 4
  MISC_FEATURE (2)..(2): Xaa at position 2 is Trp, dTrp or Val
  MISC_FEATURE (3)..(3): Xaa at position 3 is Trp, dTrp or Ser
  MISC_FEATURE (4)..(4): Xaa at position 4 is is Arg, Lys or Ser
  MISC_FEATURE (7)..(7): Xaa at position 7 is Glu or dGlu
  MISC_FEATURE (8)..(8): Xaa at position 8 is Trp, dTrp, dGlu or Glu
  MISC_FEATURE (15)..(15): Xaa at position 15 is Ile or dIle
  MISC_FEATURE (16)..(16): Xaa at position 16 is Ala or Glu
DXXXYLXXQAVREFXXWLVRGGPSSGAPPPS